(19) United States
(12) Patent Application Publication
(10) Pub. No.: US 2025/0228430 A1
(43) Pub. Date: Jul. 17, 2025

(54) MEDICAL SYSTEM, METHOD OF OPERATING A MEDICAL SYSTEM AND METHOD OF COUPLING A MEDICAL IMAGING INSTRUMENT TO A DISPLAY DEVICE

(71) Applicant: KARL STORZ SE & Co. KG, Tuttlingen (DE)

(72) Inventors: Sebastian WENZLER, Tuttlingen (DE); Clemens BÜHLER, Tuttlingen (DE); Irina GERLACH, Tuttlingen (DE); Mark SCHMELZER, Tuttlingen (DE); Gerald CLEMENS, Tuttlingen (DE)

(73) Assignee: KARL STORZ SE & Co. KG, Tuttlingen (DE)

(21) Appl. No.: 19/015,266

(22) Filed: Jan. 9, 2025

(30) Foreign Application Priority Data
Jan. 12, 2024 (DE) 10 2024 100 946.2

(51) Int. Cl. A61B 1/00; A61B 1/267

(52) U.S. Cl. CPC A61B 1/0004; A61B 1/00016; A61B 1/00045; A61B 1/267

(57) ABSTRACT

The disclosure relates to a medical system comprising a medical imaging instrument adapted to generate image data and to transmit the image data wirelessly, a display device adapted to receive image data and to generate a representation for a user, and a gesture recognition unit adapted to recognize a gesture of the user directed to the display device and to establish a coupling between the imaging instrument and the display device in accordance with the recognition of the gesture. The present disclosure also relates to a data transmission adapter, a method for retrofitting a medical imaging instrument and a method for transmitting image data. Further, the disclosure relates to a method of operating a medical system and a method of coupling a medical imaging instrument to a display device.

### CROSS-REFERENCE TO RELATED APPLICATIONS

This application claims priority of German Patent Application No. DE 10 2024 100 946.2 filed on Jan. 12, 2024, the contents of which is incorporated herein.

### TECHNICAL FIELD

The present disclosure relates to a medical system, a method of operating a medical system, and a method of coupling a medical imaging instrument to a display device.

### BACKGROUND

An endoscope is a medical imaging instrument used to examine anatomical structures in a patient's cavity. Only a relatively small imaging portion of the endoscope needs to be inserted into the patient's cavity via an access port. The internal structures can then be viewed by medical staff, such as a doctor, without the need for invasive surgery. In this manner, it is possible to visually examine body cavities, organs and other hollow structures efficiently.

Over time, different types of endoscopes have been developed to meet the needs of different medical specialties. For example, there are special endoscopes for airway management to secure the airways, in gastroenterology to examine the stomach, intestines and other parts of the digestive tract, for bronchoscopy to examine the airways, for laparoscopy to gain an insight into the abdomen to perform minimally invasive surgery, and for orthopedics to examine joints and tendons.

Endoscopes typically have a camera that images the anatomical structures and generates image data. This image data is transmitted to a display device, which generates a representation based on the captured image data. This allows the medical staff to view the anatomical structure via the display device or the treatment can be recorded for documentation purposes.

Many endoscopes have an elongated thin shaft so that the camera can be fed to and positioned in a patient's cavity. The shaft is partially fed via the access port of the cavity. For example, a bronchoscope, an endoscope for examining the lungs, is fed through the mouth or nose along the trachea to the lungs and positioned within them. At its distal end, the shaft may have an optic that collects light from the anatomical structure to be viewed. This collected light is transmitted to an image sensor of the camera, which is located approximately in a distal portion of the shaft or in a proximal handle of the endoscope to which the shaft is mounted. The image sensor converts the collected light into electrical signals and generates the image data.

Data cables are usually used to transmit the image data from the image sensor to the display device. However, cables have often proved to be a nuisance when using endoscopes. For example, there are many medical instruments and apparatus in use in an operating room or an ambulance, each of which may have cables for data transmission or power supply. This can create a mess and the cables can interfere with one another. As a result, medical staff may be restricted in their freedom of movement and the quality of treatment may be reduced. In addition, the comfort for medical staff when using endoscopes may be reduced, which may result in less acceptance for the use of endoscopes.

To overcome these limitations, wireless endoscopes are increasingly being used. Such an endoscope transmits the image data wirelessly to a display device, which generates a representation based on the image data. However, the use of wireless endoscopes creates new problems.

Endoscopes and also display devices are usually in use for several years in order to be operated economically. For example, many healthcare facilities have been using functional wired endoscopes for several years. Compatibility problems may arise if, for example, a new display device is purchased. The inventors have recognized that an older wired endoscope may not be able to be operated with the new display device. This may result in several display devices having to be purchased or old endoscopes that are still in working order having to be taken out of service.

Furthermore, problems can arise if several different wireless imaging instruments are in use at the same time or if different display devices are available. However, the inventors have recognized that an imaging instrument in use should transmit the image data to a specific display device that the medical staff has designated for use with the imaging instrument. However, it can happen that the image data is transmitted to a different, incorrect display device, which can lead to confusion as to which display device actually generates a representation based on the image data of the imaging instrument used. For example, image data can be transmitted to a display device in a neighboring operating room of a healthcare facility. This may result in the medical staff being shown a representation of an anatomical structure of the wrong patient. The inventors have recognized that this can pose a safety risk and lead to serious treatment errors. In addition, various display devices can be available in an ambulance. In emergency situations, this may mean that the display device to which the image data is actually transmitted must first be found. This can result in a critical loss of time.

Further, the inventors have recognized that the presence of multiple display devices and wireless instruments can also create problems for the process of pairing an imaging instrument to a display device or activating functions of the imaging instrument. It can be difficult to match certain wireless instruments with certain display devices. In particular, if many different wireless instruments are in use, incorrect pairing, unintended activation of functions and incorrect assignment may occur. Pairing and activation can also be problematic in time-critical situations. In such situations, it is important to be able to establish a pairing reliably, quickly and safe to operate and to activate functions so that a patient can be treated as quickly as possible.

Furthermore, it may happen that a practitioner wishes to establish a pairing while performing an action on the patient. The inventors have recognized that in such situations the practitioner may not have a free or clean hand to carry out a pairing procedure. This problem can also occur if the practitioner wants to change and/or add a display device, for example.

### SUMMARY

Based on the prior art, the aim is to enable improvements in the field of medical imaging.

Based on the prior art, the disclosure can be based on the object of improving the usability and flexibility of medical imaging instruments.

Based on the prior art, the disclosure can further be based on the object of increasing the operating safety and/or operational safety of a medical imaging instrument, in particular a wireless medical imaging instrument.

Based on the prior art, the disclosure can also be based on the object of making the operation of a medical imaging instrument safer and/or more convenient.

Based on the prior art, the disclosure can also be based on the object of simplifying the operation of a medical imaging instrument.

At least one of these objects is achieved according to the disclosure by a medical system, a method of operating a medical system and a method of coupling a medical imaging instrument to a display device as described herein and defined in the claims. In addition, further aspects of the disclosure are described which explain the disclosure.

According to one aspect, the present disclosure may provide for providing a medical system. The medical system may comprise a medical imaging instrument adapted to generate image data and comprising a plug port for connecting a data cable, and a data transmission adapter detachably connectable to the plug port and adapted to receive the image data via the plug port and to wirelessly transmit data based on the image data.

According to one aspect, the present disclosure may further provide for providing a data transmission adapter for a medical system according to the disclosure.

According to one aspect, the present disclosure may further provide for providing a data transmission adapter, in particular for a medical system according to the disclosure. The data transmission adapter may comprise a connection element detachably connectable to a plug port of a medical imaging instrument, a control unit connected to the connection element and adapted to receive image data from the connection element, and a data interface connected to the control unit and adapted to receive data from the control unit and transmit data wirelessly.

According to one aspect, the present disclosure may further provide for providing a method for retrofitting a medical imaging instrument, in particular by means of a medical system according to the disclosure and/or by means of a data transmission adapter according to the disclosure. The method of retrofitting may comprise the steps of providing a medical imaging instrument adapted to generate image data and comprising a plug port for connecting a data cable, providing a data transmission adapter detachably connectable to the plug port and adapted to receive and wirelessly transmit the image data via the plug port, and connecting the data transmission adapter to the plug port of the imaging instrument.

According to one aspect, the present disclosure may further provide for providing a method for transmitting image data, in particular by means of a medical system according to the disclosure and/or by means of a data transmission adapter according to the disclosure. The method of transmitting image data may comprise the steps of providing a medical imaging instrument adapted to generate image data and comprising a plug port for connecting a data cable, providing a data transmission adapter detachably connectable to the plug port and adapted to receive and wirelessly transmit the image data via the plug port, connecting the data transmission adapter to the plug port of the imaging instrument, generating image data by means of the imaging instrument, transmitting the image data to the data transmission adapter via the plug port, and wirelessly transmitting the image data by means of the data transmission adapter.

According to an aspect, the present disclosure may further provide for providing a method of operating a medical system according to the disclosure and/or a medical imaging instrument according to the disclosure.

These features can improve the usability and flexibility of a medical imaging instrument. In particular, these features make it possible to adapt a medical imaging instrument for wireless data transmission. This allows the medical imaging instrument to be converted from a wired imaging instrument to a wireless imaging instrument. This means that the imaging instrument can be modernized. For example, an operating room can be equipped with wireless display devices and yet old, expensive imaging instruments can still be operated. This can improve the economic operation of a healthcare facility. Old imaging instruments can therefore be compatible with new display devices and forward and backward compatibility can be achieved. The imaging instrument does not require any structural or design changes and/or adaptations to be converted to a wireless imaging instrument. The data transmission adapter can be connected and/or operated with the existing plug port. Furthermore, new imaging instruments can also be provided, which can be provided for optional wireless and/or wired image data transmission. For example, the data transmission adapter can be provided as a module, which can save production costs.

According to a further aspect, the present disclosure may provide for providing a medical system. The medical system may comprise a medical imaging instrument adapted to generate image data, a display device adapted to receive image data and generate a representation for a user, a pairing unit adapted to establish a pairing of the imaging instrument and the display device, wherein, in a paired state, the image data is transmittable from the imaging instrument to the display device, and a security unit adapted to recognize a confirmation of the pairing by the user, wherein the security unit is adapted to prevent the transmission of the image data from the imaging instrument to the display device in the absence of the confirmation of the pairing by the user.

According to one aspect, the present disclosure may further provide for providing a medical imaging instrument for a medical system according to the disclosure.

According to one aspect, the present disclosure may further provide for providing a medical imaging instrument, in particular for a medical system according to the disclosure. The medical imaging instrument may comprise a pairing unit adapted to establish a pairing of the imaging instrument to a display device, wherein, in a paired state, the image data is transmittable from the imaging instrument to the display device, and a security unit adapted to recognize a confirmation of the pairing by the user, wherein the security unit is adapted to prevent the transmission of the image data from the imaging instrument to the display device in the absence of the confirmation of the pairing by the user.

According to an aspect, the present disclosure may further provide for providing a method of operating a medical system according to the disclosure and/or a medical imaging instrument according to the disclosure.

According to an aspect, the present disclosure may further provide for providing a method of connecting a medical imaging instrument to a display device, in particular performed by means of a medical system according to the disclosure and/or a medical imaging instrument according to the disclosure. The medical imaging instrument may be adapted to generate image data, and the display device may be adapted to receive image data and generate a representation for a user. The method of connecting may comprise the steps of establishing a pairing of the imaging instrument and the display device, wherein, in a paired state, the image data is transmittable from the imaging instrument to the display device, recognizing a confirmation of the pairing by the user, and preventing the transmission of the image data from the imaging instrument to the display device in the absence of the confirmation of the pairing by the user.

These features can increase the operating safety and/or operational safety of a medical imaging instrument, in particular a wireless medical imaging instrument. A specific pairing of an imaging instrument and a display device can be achieved. In addition, it can be ensured that the imaging instrument intended for use is actually paired with a specific display device. In particular, when many different wireless apparatuses and/or instruments are in use, the targeted pairing can still be established. These features also allow a pairing to be established in a user-friendly and efficient manner. As a result, treatment errors can be prevented and a safe pairing can be established quickly and reliably in emergency situations. User confirmation provides feedback to the user that a proper, intentional pairing of a particular imaging instrument to a particular display device is established. Compared to conventional pairing, the user can therefore be more certain that the correct pairing has been established. By preventing the transmission of image data in the absence of confirmation, it is possible to prevent the unintentional transmission of image data to an unintended display device. For example, image data can be prevented from being transmitted unintentionally to a display device in a neighboring operating room. In general, this increases operational safety when operating several medical imaging instruments, in particular simultaneously.

According to a further aspect, the present disclosure may provide for providing a medical system. The medical system can be an object that has a recognition feature and that is identifiable by means of the recognition feature, a medical imaging instrument with a camera by means of which images can be captured and image data can be generated, wherein an image of the recognition feature of the object can be captured by means of the camera, a recognition unit adapted to recognize the presence of the recognition feature in the image of the recognition feature captured by means of the camera and to generate a recognition signal in accordance with the recognition of the presence of the recognition feature, and a control unit adapted to process the recognition signal and to activate at least one function of the medical system relating to the object in accordance with the recognition signal.

According to one aspect, the present disclosure may further provide for providing a medical imaging instrument for a medical system according to the disclosure.

According to one aspect, the present disclosure may further provide for providing a medical imaging instrument, in particular for a medical system according to the disclosure, in particular an endoscope. The medical imaging instrument can comprise a camera by means of which images can be captured and image data can be generated, a recognition unit adapted to recognize the presence of a recognition feature in an image of the recognition feature captured by means of the camera and to generate a recognition signal as a function of the recognition feature, and a control unit adapted to process the recognition signal and to activate at least one function of the medical imaging instrument in accordance with the recognition signal.

According to an aspect, the present disclosure may further provide for providing a method of operating a medical system according to the disclosure and/or a medical imaging instrument according to the disclosure.

According to one aspect, the present disclosure may further provide for providing a method using a medical system, in particular one according to the disclosure. The medical system may comprise an object having a recognition feature and being identifiable by means of the recognition feature, and a medical imaging instrument having a camera by means of which images can be captured and image data can be generated, wherein an image of the recognition feature of the object can be captured by means of the camera. The method may comprise the step of capturing an image of the recognition feature of the object using the camera.

These features can make the operation of a medical imaging instrument safer and/or more comfortable. Furthermore, operating safety and/or operational safety of the, in particular wireless, medical imaging instrument can be increased. For example, it can be achieved that a function can be activated without touching it. It can also be ensured that only the imaging instrument can be specifically activated. Activation based on the recognition feature can be implemented as a cost-effective, simple and efficient security function. Furthermore, existing imaging instruments can be operated with reference to the recognition feature. It can be advantageously utilized that imaging instruments at least often already have a camera. This means that no structural adaptation of already existing or conventional imaging instruments is required to provide the recognition feature and implement the recognition unit. The already existing camera can be used. Furthermore, the user can activate it himself. This ensures that a certain function of the imaging instrument is not activated unintentionally and that it is only activated in accordance with the recognition feature. For example, activating a function in certain situations may be legally questionable. If the function comprises, for example, sharp image acquisition of an object area, it may be unintentional for image acquisition to be carried out without the patient's prior consent. Furthermore, there may be legal provisions that prohibit certain features of a patient, such as their face, from being recognizable on captured images and, in particular, these images from being stored on central data stores.

According to a further aspect, the present disclosure may provide for providing a medical system. The medical system may comprise a medical imaging instrument adapted to generate image data and to transmit the image data wirelessly, a display device adapted to receive image data and to generate a representation for a user, and a gesture recognition unit adapted to recognize a gesture of the user directed towards the display device and to establish a pairing between the imaging instrument and the display device in accordance with the recognition of the gesture.

According to an aspect, the present disclosure may further provide for providing a method of operating a medical system according to the disclosure.

According to an aspect, the present disclosure may further provide for providing a method of pairing a medical imaging instrument to a display device, in particular to a medical system according to the disclosure. The medical imaging instrument may be adapted to generate image data and transmit the image data wirelessly, and the display device may be adapted to receive image data and generate a representation for a user. The method may comprise the steps of recognizing a gesture of the user directed to the display device and establishing a pairing between the imaging instrument and the display device in accordance with the recognition of the gesture.

These features can simplify the operation of a medical imaging instrument. In particular, the pairing of a medical imaging instrument with a display device can be simplified. In a time-critical situation in an operating room, operation of the imaging instrument can be simple and a representation on a preferred display device can be initiated quickly. In addition, the user can initiate pairing without having to touch an object. For example, he does not have to use his hands to operate a touch-sensitive user interface. This allows the user to initiate the pairing intuitively and conveniently. Gesture recognition makes it possible to add or switch on a display device particularly quickly. The user does not have to use the operating means provided for this purpose or initiate pairing via adjustments to display devices. For example, it may be the case that the user wants to add a specific display device so that other medical personnel can follow a procedure on it. The user can do this quickly and easily using the gesture. The fact that the gesture is directed at the display device minimizes the risk of operating errors. Spontaneous, unwanted pairing can at least be substantially prevented.

The various aspects mentioned above can enable improvements in the field of medical imaging.

When a device, unit and/or a component, in particular a medical system and/or an imaging instrument, is described in connection with various aspects of the present disclosure, it may in principle be the same device, unit and/or a component, in particular the same medical system and/or the same imaging instrument. This may mean, for example, that a medical system may have features of various aspects described herein.

The medical system may be a medical imaging system, in particular a medical endoscopy system. This may mean that the medical system is adapted for endoscopic imaging. Alternatively or additionally, the medical system may be a medical exoscopy system that is adapted for exoscopic imaging. The medical systems according to the several aspects of the present disclosure may comprise the same medical system. A medical system may have features according to several aspects of the present disclosure. In particular, in some embodiments, features of the several aspects may interact. In general, units and/or assemblies that have the same designation can represent the same unit and/or assembly. Alternatively or additionally, different units and/or assemblies can also be meant.

Associated with the medical system may be any component provided in connection with a use of the medical imaging instrument and/or the display device. For example, the medical system may comprise tools such as endoscopic forceps, suction tubes, tracheal tubes, probes and/or the like. Furthermore, the medical system may comprise at least one data processing device. The data processing device may comprise a processor, a data store, working memory, a graphics processor and/or the like. The data processing device may comprise a central data processing device and/or a computing unit, in particular a central computing unit, which is adapted to process data from a plurality of components, for example a plurality of imaging devices and/or display devices. In particular, the data processing device can be adapted to process image data. Units and/or components described herein may be implementable and/or implemented on the data processing device. The data processing device may be located in a working environment of the medical imaging instrument. This may mean that the data processing device is located in an operating room or treatment room where the imaging instrument is used. In this context, the data processing device may comprise a computer and/or a laptop. Furthermore, a central computing unit can be at least part of a data processing device.

Furthermore, the data processing device may comprise a cloud-based data processing device. A cloud-based
data processing device may be a data processing device that provides shared data processing capacity, for example in the form of servers and/or data stores.

The medical imaging instrument may comprise an exoscope and/or an endoscope. The medical imaging instrument may be adapted to be inserted into a patient's body cavity. A body cavity can comprise any body cavity and/or cavity of the patient. For example, the body cavity may comprise the nasal cavity, the oral cavity, the trachea, the lungs, the stomach, the intestines, an abdominal cavity, a thoracic cavity and/or a joint space.

In general, the medical imaging instrument can be adapted to image an anatomical object area and generate image data of the object area. The anatomical object area can refer to a part of the patient's body. The anatomical object area may comprise an anatomical structure, for example an inner wall of an intestine, an inner surface of a lung, cartilage clasps of a trachea, an inner wall of a vessel and/or the like of a patient.

The imaging instrument, in particular the endoscope, may comprise a base portion, and/or a shaft, wherein the shaft extends distally from the base portion. The base portion can comprise a handle. The shaft can be elongated and/or thin. In particular, the shaft can have a large aspect ratio. The shaft can have an outer diameter of, for example, up to 2 cm, in particular up to 1 cm, in some embodiments up to 0.5 cm. The shaft can have a length of, for example, up to 300 cm, in particular up to 150 cm, preferably up to 50 cm. The shaft can be rigid and/or flexible, at least in portions. Furthermore, the shaft may have a movable portion, in particular a movable distal end portion. “Movable” can mean that the user can bend the movable portion in a targeted manner, for example by means of a cable pull and/or by means of a motor. The shaft can be adapted to be at least partially inserted into the patient's body cavity.

To image the object area, the imaging instrument, in particular the shaft and/or the distal end portion, may have an input optic, for example one or more lenses, which is adapted to collect light, in particular object light. The light can be directed onto an image sensor. The image sensor can be adapted to generate image data based on the light. The image sensor can be arranged in the shaft, in particular in the distal end portion. In this case, the image data can be guided along the shaft to the base portion, for example by an electronic and/or optical data line. Alternatively or additionally, an image sensor may be arranged in the base portion and/or in an external imaging device.

According to some embodiments, the imaging instrument may comprise a device for wireless data transmission. In particular, the imaging instrument can be adapted to wirelessly transmit data based on the image data. Alternatively or additionally, the data transmission adapter can be connected to the imaging instrument. The imaging instrument can be adapted to wirelessly transmit data using the connected data transmission adapter. The imaging instrument together with the data transmission adapter may define an imaging instrument according to some aspects of the disclosure.

Furthermore, the imaging instrument may have an illumination device for illuminating the object area. For example, the illumination device may include an illumination means in the distal end portion. Alternatively or additionally, the illumination device can comprise a light interface by means of which external illumination light, which can be generated by a light source, can be coupled into the imaging instrument, in particular the shaft. The illumination device may further have light extraction optics, for example a diverging lens, by means of which illumination light can be extracted from the imaging instrument. Alternatively or additionally, the imaging instrument, in particular the illumination device, may comprise an illuminant. The illuminant can be arranged in the distal end portion. The illuminant can comprise at least one LED.

Components of the imaging instrument that contribute to the generation of image data, for example the input optics, the image sensor, the illuminant and/or the like, can together define a camera. The camera is adapted to capture images, in particular of an object area, and to generate image data. The term “image” can be used synonymously with the term “illustration”.

The image data may comprise analog and/or digital signals and/or digital information representing a representation, in particular of the anatomical object area. In particular, the image data, in particular visual data, can carry information about an object area. The image data can be divided into several sub-areas, in particular pixels, each of which carries, for example, color values, brightness values and/or intensity values. The image data can be transmitted wired and/or wirelessly. Furthermore, the image data can be formatted in various formats and/or it can be encrypted.

The plug port may comprise an interface by means of which the imaging instrument can be connected to other apparatuses and/or systems. The plug port can be used to transmit image data and/or information based on the image data, control signals, status signals, energy and/or the like. Furthermore, the plug port can fulfill a mechanical function. This may mean that the plug port can comprise a plug socket. The data cable can be detachably attached to the plug port. “Connecting” in this context can also comprise “mounting”. The data cable can be attached and/or connected, for example, by means of a bayonet mechanism, a clamping mechanism, a plug-in mechanism and/or the like. The plug port may have manufacturer-specific specifications and/or features. For example, the plug port can have a certain number of pins, each of which can have a specific arrangement, in particular a manufacturer-specific arrangement. A function, in particular a manufacturer-specific function, can be assigned to the individual pins. For example, a pin can be provided for a power supply and/or data transmission.

“Detachable” may mean that the data cable is not permanently attached to the imaging instrument. An example of a non-detachable data cable is a data cable soldered to a circuit board of an imaging instrument. In some embodiments, it may be provided that the data cable is permanently connected to the imaging instrument, but may also be “detachably” connected to the imaging instrument. According to such an embodiment, the data cable can be detached from the imaging instrument without having to disconnect a permanent connection, such as a soldered connection. The data cable may be detachably connected to the imaging instrument by means of a screw connection, for example. Furthermore, the plug port may be enclosed by a housing of the imaging instrument. It may be necessary to open the housing at least partially to disconnect the data cable. By “permanent” may be meant that the data cable is not connected to the imaging instrument before at least substantially every use of the imaging instrument.

The data cable may be primarily provided for data transmission, in particular image data transmission. Furthermore, the data cable may be provided for power supply and/or signal transmission. For example, the data cable may comprise a plug that is compatible with the plug port. Furthermore, the data cable may comprise a plug that is compatible with a port of the data processing device and/or the display device. The plug can comprise a USB plug, for example. Furthermore, the plug may be manufacturer-specific and/or specially designed for the medical system.

The plug port can be arranged on the base portion of the imaging instrument. According to some embodiments, the base portion is arranged at a proximal portion, in particular end, of the base portion. Conventional, wired imaging instruments often have a plug port. The data transmission adapter can be adapted to connect to a plug port of a conventional, wired imaging instrument.

In particular, the data transmission adapter is an independent apparatus that can be handled completely separately. In particular, the data transmission adapter is designed separately from the medical imaging instrument, to whose plug port it is detachably connected. In particular, the data transmission adapter is not part of said medical imaging instrument. The data transmission adapter may comprise an adapter housing. The adapter housing can completely enclose the components of the data transmission adapter. Said imaging instrument may have an instrument housing. The instrument housing can completely enclose the components of the imaging instrument. The adapter housing and the instrument housing can be separate components.

The data transmission adapter may comprise the connection element which is detachably connectable to the plug port, the control unit which is connected to the connection element and which is adapted to receive the image data from the connection element, and the data interface which is connected to the control unit and which is adapted to receive data from the control unit and to transmit data wirelessly. The data interface can, for example, comprise a WLAN module, a Bluetooth module and/or the like. In principle, the data interface can give the data transmission adapter a wireless data transmission capability.

The data transmission adapter, together with the imaging instrument in a connected state, can define an imaging instrument. An “imaging instrument” may comprise an imaging instrument without a connected data transmission adapter and/or an imaging instrument with a connected data transmission adapter. An imaging instrument without a connected data transmission adapter may comprise a wired and/or a wireless imaging instrument. For example, a wireless imaging instrument may comprise an assembly that may have features of the data transmission adapter. For example, a wireless imaging instrument can be adapted to wirelessly transmit data based on the image data. The data transmission adapter can give a wired imaging instrument functions that a wireless imaging instrument has.

The data transmission adapter can generally function similar to a USB WLAN adapter. A USB WLAN adapter can be plugged into a USB port on a computer to enable wireless data transmission. This extends the wireless data transmission capability of the computer. Similarly, the data transmission adapter extends the wireless data transmission capability of the imaging instrument. In particular, the data interface can be provided for providing the wireless data transmission capability.

The data transmission adapter can be adapted to simulate and/or replace and/or functionally map a data cable. This can mean that a data transmission can be carried out on the imaging instrument side as if a data cable is connected, even though the data transmission adapter is connected. The data transmission adapter can therefore receive the image data via the plug port, in particular in the form of a data cable. The data transmission adapter may comprise the connection element, in particular a plug portion, which is compatible with the plug port. The connection element can be designed similarly to the plug of a data cable. For example, the connection element may have the mounting mechanism that is adapted to mount the data cable. The data transmission adapter can be connectable and/or mountable in the same way as the data cable. Alternatively or additionally, the data transmission adapter may comprise an adapter mounting mechanism adapted to mount the data transmission adapter to the imaging instrument. To adapt the medical imaging instrument for wireless data transmission, a user can disconnect a data cable connected to the plug port and insert the data transmission adapter into the free plug port. The data transmission adapter can subsequently have all the functions that enable wireless data transmission, wherein the data transmission adapter receives the image data via the plug port.

“Wireless transmitting” can mean any data transmission in which there is no physical connection directed at a data transmission between the transmitter and the receiver. However, a physical connection may also exist, for example in the form of a power supply. The data interface of the data transmission adapter can be provided for this purpose. A suitable data interface can be provided on the receiver side. For example, the data transmission adapter can be integrated into a wireless network, such as a WLAN network. The image data, general data and/or data based on the image data can be transmitted to a router. Furthermore, the image data, general data and/or the data based on the image data can be transmitted wirelessly via a Bluetooth connection, an NFC connection, an infrared connection, a wireless USB connection and/or the like.

The data based on the image data can also be image data. This data may comprise image data in a different format. The data based on the image data can comprise, in particular, the visual information of the image data relating to the object area. For example, the image data can be collectively transmittable wirelessly as data. If there are short interruptions in data transmission, for example, the data can be transmitted collectively. Furthermore, the image data can be filtered and/or processed before it is transmitted. Image data can refer in particular to image raw data. The data based on the image data can be processed image data.

The control unit may comprise a computing unit, in particular a processor and/or a microprocessor, a data store, a working memory, a graphics processor and/or the like. The control unit may be designed as an integral electronic component, for example on a common circuit board. The data interface can be designed together with the control unit. For example, the data interface can be arranged on the common circuit board or can be permanently connected thereto. The data interface can comprise a WLAN module and/or a Bluetooth module, for example. The control unit can be adapted to process the image data. In general, the control unit can process the image data such that data based on the image data can be transmitted wirelessly via the data interface. In some embodiments, the image sensor may also be integrally formed with the control unit and/or permanently connected thereto. According to some embodiments, the medical imaging instrument may comprise the control unit and the data interface. Such imaging instruments can be adapted for wireless data transmission without the data transmission adapter.

Imaging instruments that are not adapted for wireless data transmission can be adapted for wireless data transmission using the data transmission adapter. This may mean that these imaging instruments can be retrofitted. Imaging instruments retrofitted with the data transmission adapter can be adapted for wireless data transmission. The data transmission adapter can be permanently connected to the imaging instrument. This may mean that the adapter does not have to be detached from the imaging instrument after it has been used.

Retrofitting can comprise connecting the data transmission adapter. The connecting may comprise permanent connecting. For example, the data transmission adapter can be connected, in particular screwed and/or clamped, to a housing of the imaging instrument and/or arranged at least partially within the housing. In some embodiments, the adapter housing can be connectable and/or mechanically couplable to the instrument housing. Furthermore, “connecting” may comprise plugging the data transmission adapter into the plug port.

A display device can generally be understood as an apparatus comprising a screen. An image of the object area can be represented on the display device, in particular the screen. For example, the user can see and/or evaluate the object area on the display device based on the representation of the image data. The display device may comprise a computer screen, a screen in an operating room, a mobile terminal and/or the like. A mobile terminal may comprise a tablet, a laptop, a smartphone and/or a portable screen. The portable screen can be provided for use in an ambulance, for example, wherein various imaging instruments can be coupled to the screen.

The representation can depict information, in particular visual information, about the object area and make it available to the user. The representation can be based on the image data. The image data can be editable for representation. For example, analog and/or digital image filters, in particular smoothing filters, color filters, pass filters, can be applied to the image data.

“Pairing” can be understood as a process in which two apparatuses are connected to one another in order to exchange information and/or data and/or share a function. In an unpaired state, data cannot be transmitted from the imaging instrument, in particular to the display device. In a paired state, data can be exchanged and, in particular, data based on the image data can be transmitted. Furthermore, a function of the imaging instrument can be controlled in a paired state. If the imaging instrument has an illuminant, for example, this can be activated in a paired state via an external user interface. In the paired state, operating status data can be exchanged. For example, it may be possible to transmit information relating to the operational readiness of a camera of the imaging instrument. If the device is not ready for use, for example, it may not be possible to generate image data and/or transmit image data.

The imaging instrument, the display device and/or a data processing device may comprise the security unit. The security unit can comprise a virtual component of the medical system and/or be adapted to detect an unwanted and/or unintentional pairing of an imaging instrument with a display device. The imaging instrument, the display device and/or a data processing device may comprise at least one controller and/or at least one computing unit that implements the security unit. The security unit may have dedicated circuitry and/or be embodied at least partially as part of a programming in general circuitry of the imaging instrument, display device and/or data processing device. The imaging instrument and/or the display device may at least partially comprise the security unit. Furthermore, the imaging instrument and the display device can comprise the security unit together. The security unit can be used to ensure that only a desired and/or intended pairing is established. This means that only one intended pairing of a display device and an imaging instrument is established. If the security unit detects an unintentional pairing of an imaging instrument and a display device, the security unit can prevent data transmission, in particular image data transmission.

The security unit can decide whether an existing pairing is an intentional pairing in accordance with the user's confirmation of the pairing. If there is no confirmation, the security unit can decide that the current pairing is an unintentional pairing and prevent the image data from being transmitted. The absence of confirmation may in particular mean the absence of confirmation. This may mean that the user must actively confirm a coupling and/or pairing in order for image data to be transmittable. According to some embodiments, information and/or data can be transmitted despite the absence of the confirmation. For example, device status data and/or device identification data can be transmitted.

If there is no confirmation, the display device can inform the user, for example, about a pairing attempt and/or an unsuccessful pairing. If image data transmission is prevented, the image data can still be generated. These can be stored temporarily on a storage device of the imaging instrument. Temporarily stored image data can be transmitted to the display device and/or the data processing device when a pairing is subsequently confirmed.

Confirmation of the pairing can comprise a user input. Confirmation can, for example, involve generating a confirmation signal and/or changing the state of a confirmation signal. For example, the security unit can use the confirmation signal to decide whether a confirmation has been received. If the confirmation signal changes, the security unit can decide that there is a confirmation.

The security unit can also comprise a user interface, in particular an input device. For example, the display device can comprise a touch-sensitive screen. An operable field can be displayed on the touch-sensitive screen which, when actuated, requires confirmation of the pairing.

The pairing can be a temporary connection between two apparatuses. The pairing can be established and/or released. When the pairing is established, the connection can be established for the first time and/or re-established. If the pairing is re-established, information from a previous pairing can be used to establish the pairing. If the pairing is released, the connection can be disconnected in such a way that data can no longer be exchanged. Releasing the pairing may comprise storing information about the pairing and/or about the paired apparatuses. For example, a device identification, in particular a device identification number, and/or a connection protocol can be stored.

The object can comprise any, in particular movable, object and/or any arrangement. In some embodiments, the object may be usable in conjunction with the medical imaging instrument. For example, the object may be usable in conjunction with the imaging instrument when performing a diagnostic and/or therapeutic action. For example, the object may comprise a medical instrument, in particular a scalpel, tweezers, scissors, a hook and/or the like, another imaging instrument and/or a display device. Furthermore, the object may comprise, for example, a data cable, a data processing device, a data sheet, a form, an ambulance and/or the like. In addition, the object can be assigned to an area of a healthcare facility, for example an operating room and/or a treatment room. For example, a diagnostic and/or therapeutic action may be performed in an operating room using the imaging instrument. The object may comprise a wall, a door, a treatment table, a sign and/or the like. If the recognition feature of the object has been identified, it can be concluded, for example, that a diagnostic and/or therapeutic action is being and/or has been performed in the corresponding operating room. In principle, the object can act as an assignment aid so that an area of a building can be identified, for example. Alternatively or additionally, the object can be identified as such.

The recognition feature may have a consciously and/or deliberately generated and/or intended characteristic. It may differ from a feature of an object that is integral to that object and/or would already be present without the recognition feature being provided. This can mean that the recognition feature can be added to the object. For example, the recognition feature may be different from a conspicuous color, shape, surface texture and/or the like of the object. In this context, the recognition feature can be, for example, a pattern, a color, a color sequence, a lettering, a number and/or a combination of numbers and/or the like, which can be added to the object in a targeted manner for the purpose of recognizing and/or identifying the object. For example, a vehicle license plate can be a recognition feature of the object vehicle, but not the shape, existing damage and/or color of the vehicle. Furthermore, the recognition feature may comprise a representation that can be generated and/or displayed for the purpose of recognizing and/or identifying the object. In some embodiments, the recognition feature may not be recognizable and/or detectable by a human. For example, the recognition feature may comprise a rapid sequence of images, wherein individual images are at least substantially not processable by a human.

“Identifiable” can be understood to mean that the object is specifically and/or non-specifically identifiable. Specifically can mean that only the object that has the recognition feature is identifiable. Non-specifically can mean that a type of object is identifiable. For example, if the object is a pair of forceps, it is possible to specifically identify precisely this pair of forceps and/or a pair of forceps of the same type as the pair of forceps in question. Furthermore, the object can be identifiable by the medical system, in particular by the recognition unit and/or the data processing device. The system can recognize that the object is present.

The display device, the data processing device and/or the imaging instrument may at least partially comprise the recognition unit. The recognition unit can, for example, be implemented at least partially on a computing unit of the imaging instrument. Alternatively or additionally, the recognition unit can be implemented at least partially on the display device and/or in the cloud. For example, the computing power of a server can be used to recognize the presence of the recognition feature. The imaging instrument, the display device and/or a data processing device may comprise at least one controller and/or at least one computing unit that implements the recognition unit. The recognition unit may have dedicated circuitry and/or may be embodied, at least partially as part of a programming in general circuitry of the imaging instrument, the display device and/or the data processing device.

The recognition unit can recognize the presence of the recognition feature by means of pattern recognition, text recognition and/or image recognition. Recognition can be based on a mathematical calculation rule and/or an algorithm, for example using artificial intelligence, machine learning, deep learning and/or a neural network, in particular an open, closed, single-layer and/or recurrent neural network and/or a combination thereof. Furthermore, digital filters may be applicable to at least one image of the recognition feature to recognize the recognition feature. A brightness, a polarization, a wavelength and/or the like can be filterable in order to recognize the recognition feature.

The recognition signal may comprise an analog and/or a digital signal. The recognition signal may comprise a switching signal that can be switched between two values, such as zero and one. If the switching signal assumes the value one, this can signal the presence of the recognition feature.

The recognition unit can be connected to the control unit. In some embodiments, the recognition unit and the control unit may be implemented together on a computing unit, which may in particular be arranged on the imaging instrument. The control unit can in principle control a function of the imaging instrument based on control signals and/or the recognition signal. For example, the control unit can control a function of the camera, such as an image acquisition process, and/or a function of the imaging instrument, such as local storage and/or transmission of image data. “Control” can also mean activating the function. This can mean that a process is initiated and/or a component is switched on. For example, the illuminant, in particular the LED, can be switched on.

The control unit can be adapted to process analog and/or digital signals. For example, the control unit can make decisions based on these signals. For example, when the control unit processes the recognition signal, the control unit can set the recognition signal in conjunction with other signals, in particular control signals and/or status signals, and make a decision as to whether and when to activate the function related to the object. Status signals can, for example, relate to the operational readiness of other components of the medical system. For example, it may be desired that the function related to the object is activated with a time delay in certain situations. In this case, the control unit can process a corresponding status signal together with the recognition signal.

The function of the medical system related to the object can be a function that is directly related to the object. The function can be activated depending on the presence of the object. The function can be a function of the object itself, for example. Alternatively or additionally, the function can include the object and/or the function can be assigned by the object. For example, if the object identifies a specific operating room, the function related to the object can comprise storing information about the operating room. For example, it can be used to track which medical instruments were used in this operating room and/or to register when and/or where a certain action was performed. The presence of the object can also be stored. For example, information about medical instruments can be stored. Information about users can also be stored. In such a case, the object can be a name tag and/or another recognition feature of the user, for example. Furthermore, if the object can be switched on, it can be switched on in accordance with the recognition signal. For example, a light source can be switched on to generate external illumination light. In addition, an imaging mode of the imaging instrument can be switchable in accordance with the recognition signal. In an imaging mode, for example, a resolution can be higher. It is possible to switch to this imaging mode after a recognition feature has been recognized on a surgical instrument. If a specific surgical instrument is recognized, the imaging mode can therefore be activated with a higher resolution so that the user can view an object area in which a procedure is to be performed in greater detail.

A function of the imaging instrument may comprise a brightness of the illumination device, a recording function, a storage function, an image acquisition rate, a data transmission function, and/or the like. For example, wireless data transmission can be activated, a recording can be started and/or an additional illuminant can be switched on.

The display device, the data processing device and/or the imaging instrument may at least partially comprise the gesture recognition unit. The gesture recognition unit can, for example, be implemented at least partially on a computing unit of the display device. Alternatively or additionally, the gesture recognition unit can be implemented at least partially in the cloud. For example, the computing power of a server can be used to recognize the gesture. The imaging instrument, the display device and/or a data processing device may comprise at least one controller and/or at least one computing unit that implements the gesture recognition unit. The gesture recognition unit may have dedicated circuitry and/or may be embodied at least partially as part of a programming in general circuitry of the imaging instrument, the display device and/or the data processing device.

The gesture recognition unit can recognize the gesture using pattern recognition and/or image recognition. The gesture recognition can be based on a mathematical calculation rule and/or an algorithm, for example using artificial intelligence, machine learning, deep learning and/or a neural network, in particular an open, closed, single-layer and/or recurrent neural network and/or a combination thereof.

The gesture recognition unit may comprise a camera adapted to image an object area comprising the user and/or the imaging instrument and to generate image data of this object area. The user and/or the imaging instrument can be observed using this image data and/or a movement of the user and/or the imaging instrument can be determined. For example, an arm movement, a head turning movement and/or a body posture can be determined. It may also be possible to determine a direction in which the imaging instrument is pointed and/or held. Furthermore, the gesture recognition unit may comprise a marker, in particular an optical marker and/or an electromagnetic marker, arranged on the imaging instrument. A measuring device, for example the camera and/or an electromagnetic measuring device, of the gesture recognition unit can be used to determine a movement of the marker and a gesture can be recognized based on the determined movement. A movement can be determined in a similar way to a surgical navigation system.

The gesture may comprise a deliberate body movement explicitly performed to establish the pairing and/or a body posture explicitly assumed to establish the pairing. This does not include, for example, tracking the direction of the user's gaze and creating a representation on a display device in the direction of the user's gaze. The gesture may comprise, for example, pointing a hand and/or finger at a display device, aligning the imaging instrument in the direction of the/a display device, and/or turning and/or tilting the head in the direction of the/a display device.

The gesture recognition unit can be adapted to recognize the gesture directly. This can mean that the gesture is recognizable based on the user's behavior. Alternatively or additionally, the gesture recognition unit can be adapted to recognize the gesture indirectly.

This can mean that the gesture can be recognized by a movement of the imaging instrument and/or another object. The movement of the imaging instrument and/or the other object can also be used to infer the movement and/or gesture of the user.

In general, it may be irrelevant for some features of the present disclosure whether the imaging instrument is adapted for wireless (image) data transmission by means of the data transmission adapter or whether the imaging instrument is already adapted for wireless (image) data transmission without a data transmission adapter connected.

The data transmission adapter may comprise an energy storage adapted to provide energy to the medical imaging instrument during operation via the plug port. The data transmission adapter can be operated completely wirelessly. Not only the data transmission, but also the energy supply can be wireless. The energy storage can, for example, comprise a replaceable battery and/or a rechargeable accumulator, in particular a lithium-ion accumulator. The energy storage may have a capacity that is at least substantially sufficient to power the imaging instrument during operation for several hours, for example for at least substantially two, three, four, five and/or more than five hours.

The data transmission adapter can also comprise a charging interface that can be used to charge the energy storage. Advantageously, the data transmission adapter can be continuously and/or permanently connected to the plug port. The energy storage and/or data transmission adapter does not need to be removed or disconnected for charging. Alternatively or additionally, the data transmission adapter can comprise a quick energy storage replacement device, by means of which a new energy storage can be provided quickly and easily during operation, for example if the energy storage is discharged.

The charging interface can also be adapted for inductive energy transmission. Charging the energy storage can be carried out more conveniently and less error-prone. Fewer fragile components that could be damaged need to be provided. In addition, the data transmission adapter can be provided with a simpler fluid-tight and/or gas-tight design. This may effect that the data transmission adapter can be provided autoclavable and/or disinfectable with less technical effort. The inductive power transmission may in particular comprise inductive charging and/or enable wireless electrical power transmission from an energy source to the data transmission adapter and/or the imaging instrument. A transmitter, in particular a charging station, can be provided, which can be provided in particular in a storage device and/or charging device for the imaging instrument and/or the data transmission adapter. The transmitter can be adapted to generate an alternating magnetic field. The data transmission adapter, in particular the charging interface, can comprise a coil which detects the alternating magnetic field of the transmitter in a charging position. The changing magnetic field can generate an electrical voltage in the coil, in particular by means of induction.

Alternatively or additionally, the charging interface can be adapted for detachably connecting a charging cable. This means that the energy storage can be charged using a charging cable. Charging using a charging cable can be provided as a simple, cost-effective and/or space-saving charging option for energy storage. The charging cable can be connected during operation should the energy storage have a charge level in a critical range, for example below 20%, in particular below 15%, preferentially below 10%.

In addition, the data transmission adapter can comprise a plug port for connecting a data cable. This creates redundancy and ensures safe and reliable operation in the event of a network and/or wireless pairing failure. In addition, a data cable can be connected that is adapted to transmit image data and/or data based on image data to a display device that is not adapted for wireless data transmission. This means that an older display device can be used, for example, and operation of the imaging instrument can be achieved with display devices that were compatible before the data transmission adapter was connected. The data transmission adapter can therefore only add a function to the imaging instrument, but not impair the original function, in particular the wired data transmission, at least substantially.

Furthermore, the design of the plug port of the data transmission adapter may correspond to a design of the plug port of the imaging instrument. Compatibility with the older display device can be achieved in a particularly simple manner. The imaging instrument can optionally be operated wirelessly and/or as if the data transmission adapter is not connected.

In general, forward compatibility with wireless display devices and backward compatibility with wired display devices can be achieved by providing the plug port, in particular the plug port of the same type.

In addition, the data transmission adapter can be adapted to be at least partially disinfected and/or autoclaved. Advantageously, the data transmission adapter can be reconditioned after use and/or prepared for a new use. The data transmission adapter can be designed to be fluid-tight and/or gas-tight, in particular in a connected state.

Furthermore, the medical system may be adapted to be at least partially disinfected and/or autoclaved in a state in which the data transmission adapter is connected to the medical imaging instrument. Advantageously, the data transmission adapter does not need to be disconnected and/or removed from the imaging instrument for reconditioning the medical system. The data transmission adapter can therefore be permanently and/or continuously connected. At least partially may mean that the data transmission adapter can be disinfected and/or autoclaved together with the medical imaging instrument.

In addition, the data transmission adapter may comprise a sealing device which is adapted to seal the plug port of the imaging instrument from an environment, in particular in a fluid-tight and/or gas-tight manner, in a state in which the data transmission adapter is connected to the medical imaging instrument. The sealing device can be provided easily and/or cost-effectively, in particular together with the connection element. The sealing device can, for example, comprise a labyrinth seal, a diaphragm seal, a sealing ring, in particular an O-ring, and/or the like. The connection element and the plug port can together form the sealing device. For example, the plug port may have a sealing portion by means of which the imaging instrument is sealed from an environment, in particular in a gas-tight and/or fluid-tight manner, in a state in which a data cable is connected to the imaging instrument. The sealing device of the data transmission adapter can be provided such that it interacts with the sealing portion of the plug port for sealing.

In addition, the data transmission adapter can be adapted to prepare the image data for wireless transmission. Data transmission can be made more secure, less error-prone, faster and/or more efficient.

For example, “preparing” can mean that the image data is encrypted, compressed, formatted, packaged, fragmented and/or cleansed. Encryption can be used to make data transmission more secure and to better protect sensitive image data from unauthorized access. This can be particularly important for image data of a patient that was generated in a medical environment. By compressing and cleansing, a larger amount of data can be transmitted per unit of time. Furthermore, faulty image data can be recognized and/or repaired. The image data can be prepared for wireless transmission by formatting, packaging and/or splitting. In particular, it may be possible to generate data based on the image data, wherein the data is suitable for wireless transmission. For example, a Bluetooth connection and/or a WLAN connection may require data to be packetized, have a certain format and/or be encrypted in a certain way. The image data can at least be prepared according to these requirements. Data that is transmitted can be the image data and/or data based on the image data. The designation of both forms of data transmission can be used interchangeably and/or mean the same thing. If, for example, it is required that image data be transmitted, this may mean that data based on the image data is transmitted. It is understood that the same image data generated by the camera is not necessarily transmitted unchanged.

Furthermore, the data transmission adapter can be adapted to transmit the image data directly to a mobile terminal wirelessly. The imaging instrument can be operated with the mobile terminal. Flexibility and/or versatility of the imaging instrument can be improved. The mobile terminal may comprise, for example, a smartphone and/or a tablet. “Directly” can mean that the image data is sent by means of the data transmission adapter and received by means of the mobile terminal, in particular without another device such as the display device being interposed. For example, such data transmission can be carried out using Bluetooth and/or Wi-Fi Direct.

In addition, the plug port may be manufacturer-specific. This can mean that a number and/or arrangement of connection elements, in particular pins, is standardized for a manufacturer and differs from manufacturer to manufacturer. Furthermore, the shape and/or mounting mechanism of the plug port can be manufacturer-specific. This can mean that only manufacturer-specific plugs are compatible with the plug port and/or that the control or pin assignment is manufacturer-specific. In particular, plugs may be certified for certain plug ports and only a certified plug with a certain plug port may be legally permitted for a medical application. The connection element of the data transmission adapter can have such a certification and/or a manufacturer-specific shape and/or pin assignment.

In addition, the data transmission adapter can be adapted to receive at least one control signal wirelessly and transmit the control signal to the imaging instrument via the plug port. The imaging instrument may be at least substantially wirelessly operable. In some embodiments, at least substantially all functions of the imaging instrument may be wirelessly controllable and/or activatable. In addition to control signals, other signals, such as status signals, can be transmitted wirelessly.

In addition, in a state in which the data transmission adapter is connected to the medical imaging instrument, the data transmission adapter and the imaging instrument can together form an imaging instrument that can be handled by a user as a single instrument. Operating the imaging instrument can be intuitive. In particular, the data transmission adapter may be ergonomically shaped and/or may at least substantially not interfere with an ergonomic shape and/or feature of the original imaging instrument that has the plug port and is intended for wired data transmission. For example, the data transmission adapter may be comparatively small, in particular in relation to the proximal handle of the imaging instrument. Furthermore, the data transmission adapter can accommodate and continue a shaping, in particular of the handle, of the data transmission adapter. A transition from the handle to the data transmission adapter can be shaped at least substantially harmoniously. The data transmission adapter can, for example, continue and/or extend the handle. This may mean that the data transmission adapter has a handle portion. In the connected state, the user can therefore use the imaging instrument as if the imaging instrument was already adapted for wireless data transmission without the provision of the data transmission adapter.

In addition, the data transmission adapter can comprise an operating element by means of which the data transmission adapter can be set to a pairing mode for establishing a pairing with a display device. This can improve user-friendliness and make the data transmission adapter and/or imaging instrument easier to operate. The operating element can, for example, comprise a push button, a rotary knob, in particular a touch-sensitive control panel such as a touch screen and/or a touchpad. In the pairing mode, a pairing can be initiated, in particular from the data transmission adapter, and/or confirmed. For example, the data transmission adapter can request a pairing and/or the data transmission adapter can be asked for confirmation to perform a pairing. For confirmation, it may be sufficient for the user to press the operating element. Furthermore, to prevent incorrect operation, it may be necessary to press the operating element for several seconds to establish, initiate and/or confirm a pairing. The operating element can be at least substantially a part of the/a pairing unit and/or the/a security unit.

In addition, the medical system may comprise a further medical imaging instrument, wherein the data transmission adapter is optionally connectable to the imaging instrument or the further imaging instrument. Various imaging instruments can be provided for wireless data transmission in a simple manner. This means that a data transmission adapter does not have to be provided for each imaging instrument. In particular, a standardized plug port can ensure broad compatibility of the data transmission adapter.

In addition, preventing the transmission of the image data may comprise releasing the pairing of the imaging instrument and the display device. Advantageously, this can ensure that a pairing of another imaging instrument is not prevented and/or blocked and at the same time it can be ensured that image data is not transmitted erroneously. The pairing can be released after device information and/or information about a pairing attempt has been transmitted. Furthermore, a notification can be shown on the display device to inform the user of a pairing attempt.

Furthermore, the security unit can be adapted to issue a request to the user to confirm the pairing. This provides a safe to operate and simple pairing process. In principle, the request can comprise any information and/or any indication to the user that they should carry out the confirmation. For example, the request may comprise a vibration alarm, a sound, a flashing LED, a color change of a light indicator, a prompt text in a control panel, in particular in the control panel of the data transmission adapter, and/or the like.

Furthermore, the display device can be adapted to display the request to the user. Advantageously, the user has immediate feedback as to which specific display device the imaging instrument is pairing with. A particularly safe to operate medical system can be provided. Furthermore, the user often has the display device with which he wants to couple the imaging instrument in view anyway. This makes pairing intuitive, simple and safe, as the user does not have to look away from the display device. A request representation can be generated on the display device and/or an overlay can be superimposed on a representation of the display device. The request can also display information about the imaging instrument to be paired. For example, an imaging instrument name, a symbol identifying the imaging instrument, an identification number and/or the like can be displayed together with the request.

In addition, the medical imaging instrument may comprise an operating element and the confirmation of the pairing may comprise an actuation of the operating element. Simple and convenient pairing can be achieved for the user. The operating element can have at least substantially similar features to the operating element of the data transmission adapter. Furthermore, the operating element may comprise the operating element of the data transmission adapter. This can mean that when the data transmission adapter is connected, the operating element of the data transmission adapter comprises the operating element of the imaging instrument. Actuating can comprise turning, pressing, tapping and/or the like.

Furthermore, the confirmation of the pairing may comprise an actuation of the operating element over a predetermined minimum confirmation time period, wherein the confirmation time period is preferentially at least three seconds long and preferably at least five seconds long. This can at least substantially reduce the risk of unintentional pairing. At the very least, however, the probability of unintentional pairing can be reduced.

Furthermore, instructions for confirmation can be displayed together with the request and/or the request can comprise the instructions. For example, information about the minimum confirmation time period can be displayed. Alternatively or additionally, confirmation of the pairing can comprise actuating the operating element via an actuation sequence for which instructions can be displayed. Confirmation can, for example, comprise pressing the operating element three times.

In addition, the security unit can be adapted to allow the image data to be transmitted from the imaging instrument to the display device when the pairing is confirmed. It can be ensured that image data is only transmitted after confirmation, allowing the user to check and ensure that image data is only transmitted to the correct display device. Confirmation can therefore be made a condition for the transmission of image data.

In addition, the display device may have a recognition feature and may be identifiable by the recognition feature, and confirmation of the pairing may comprise capturing an image of the recognition feature using the imaging instrument. Advantageously, this gives the user direct confirmation that the correct display device is paired. Furthermore, this eliminates the need to provide an operating element, since the camera of the imaging instrument can be used for confirmation. Pairing can be particularly reliable, secure, convenient and quick to carry out. The recognition feature may correspond to the recognition feature of an aspect of the present disclosure. In general, these features may refer to at least one other aspect of the present disclosure. For a more extensive description, please refer to at least one other aspect.

Furthermore, the display device can be adapted to display the recognition feature. The display device and the imaging instrument can thus be coupled without the need for structural and/or design measures. Functions of the display device and the imaging instrument that are already provided can be used. The recognition feature can, for example, be displayed by means of an overlay that can be superimposed on a representation. Furthermore, a separate representation can be generated that comprises the recognition feature. In general, these features may refer to at least one other aspect of the present disclosure. For a more extensive description, please refer to at least one other aspect.

In addition, a first device identification can be assigned to the imaging instrument, a second device identification can be assigned to the display device, an assignment rule can be stored in the pairing unit, which defines combinations of different device identifications as permissible or impermissible, and the pairing unit can be adapted to compare the first device identification with the second device identification using the assignment rule and to establish or prevent pairing of the imaging instrument and the display device in accordance with the application of the assignment rule. This can improve operational safety. It can be ensured that only desired pairings of imaging instruments and display devices are established. For example, only pairings according to the assignment rule are established. The device identification can be adapted to uniquely assign and/or identify an apparatus. The device identification may have an at least substantially unique identifier for a particular apparatus. For example, the first and/or the second device identification may comprise a device identification number by which the imaging instrument and/or the display device is identifiable and/or assignable. The assignment rule can set device identifications in relation to one another. The assignment rule can be updated before each pairing process. For example, the assignment rule can be retrieved from a cloud storage before a pairing process and/or during a pairing process. The assignment of different apparatuses and/or instruments can thus be managed centrally. The permissibility of combinations can be managed and/or controlled by means of a central data service, in particular by means of a cloud. For example, a combination can be set in a healthcare facility inventory software, which sends the combination to the pairing unit via the central data service. Furthermore, the user can create the assignment rule using a list selection. For example, the user can create the assignment rule on the display device and/or a mobile terminal, in particular using a list selection. This newly created assignment rule can be stored in the pairing unit and/or the assignment rule that is already stored in the pairing unit can be updated using the newly created assignment rule. Only one combination of device identifications can be stored for an imaging instrument. This ensures that image data can only be transmitted to a specific display device. Alternatively or additionally, several combinations can be stored. This allows, for example, a multi-layer pairing safety procedure to be carried out. Certain combinations may be excluded, and a permitted combination can be confirmed in a further confirmation step.

In addition, the security unit may be adapted to determine a user authorization of the user, compare the user authorization with a predetermined required authorization, and prevent transmission of the image data from the imaging instrument to the display device if the user authorization does not reach the predetermined required authorization. This can prevent unauthorized users from using the imaging instrument. Furthermore, a user authorization can be activated centrally and thus, for example, a user-based payment system and/or registration system can be implemented. The user can, for example, have usage activated for a fee and/or be authorized to use the imaging instrument over a period of time via a subscription. User authorization can be purchased and/or activated via a mobile terminal such as a smartphone, for example. The purchase and/or activation can be carried out in a manner similar to a purchase in an app store, for example. Alternatively or additionally, the authorization can also be carried out by a central device management service of a healthcare facility. This can help to achieve better control and/or oversight of the use of equipment, in particular imaging instruments. The term “not reached” can be understood to mean that the authorization comprises authorization for individual functions. For example, authorization for individual functions of the imaging instrument may be available for purchase. A user may be authorized to generate and/or transmit SD image data, for example, but may wish to generate and/or transmit HD image data. In this case, the authorization does not reach the specified required authorization. However, the user can purchase the required authorization, for example, again using the mobile terminal in the manner of a purchase in an app store.

Furthermore, the medical system may comprise a storage unit in which information about the pairing of the imaging instrument with the display device and/or the confirmation of the pairing by the user is stored. This makes it possible to trace which imaging instrument was paired with which display device. The information may comprise, for example, a pairing attempt, at least one device identification, a user identification by which the user of the imaging device is identifiable, a pairing duration, a usage duration, an amount of image data, a usage type and/or the like. The storage unit may at least partially comprise a storage unit already described. In one embodiment, the storage unit may comprise and/or be in communication with a cloud-based storage unit. This means that the information can be stored and/or accessed centrally. The information may be evaluated, in particular for statistical purposes, for example for analysis and/or billing purposes.

In addition, the object may comprise a display device adapted to receive image data and generate a representation for a user. A display device is usually provided for operating the imaging instrument. Advantageously, the display device can be integrated into the activation process. The display device may correspond to the already described display device of one aspect of the present disclosure. In general, these features may refer to at least one other aspect of the present disclosure. For a more extensive description, please refer to at least one other aspect.

In addition, the display device may be adapted to display the recognition feature. Advantageously, the recognition feature can be generated and/or displayed on demand. Flexible activation can be carried out. In particular, the recognition feature can be displayed on demand. It is only displayed and/or only present when it is needed. No installation space needs to be provided for the recognition feature and it can meet current requirements. For example, the recognition feature can be provided in an application-specific and/or imaging instrument-specific manner. It can be generated on demand and have at least one feature specific to an activation process. This means that when an activation is to be performed, a recognition feature can be generated, wherein the recognition feature is specific to the activation to be performed and/or has the specific feature. For example, if the recognition feature comprises a numerical code, the numerical code can be generated specifically for a particular activation process. The recognition feature can be displayed by means of an overlay that can be superimposed on another representation. Further, the recognition feature may comprise a representation that at least substantially fills an entire display portion, such as the screen of the display device.

Furthermore, the action related to the object can be a pairing of the imaging instrument with the display device. This allows the pairing to be carried out particularly simple, fast, reliable, secure and/or convenient. Furthermore, only components that are already available can be used for the pairing. Advantageously, it can be utilized that the imaging instrument has a camera anyway and that the display device is designed for displaying images. It may be possible to provide for features of the display device and/or the imaging instrument to work together. Design and/or manufacture can be carried out more cost-effectively. For example, if the user wants to pair the imaging instrument with a particular display device, the user can cause the recognition feature to be displayed on the particular display device to be paired. The user can capture this recognition feature with the imaging feature to be paired. This ensures that the two correct apparatuses are paired with one another. The user has immediate feedback that the two correct apparatuses are being paired with one another. If, for example, the recognition feature is not displayed on the particular display device, the pairing cannot be performed and/or the user has immediate feedback about a problem with the pairing and/or assignment of display devices.

In addition, the medical imaging instrument can be adapted to capture the image of the recognition feature of the object in a recognition mode and/or to generate associated image data such that at least image areas which do not contain the recognition feature are unrecognizable. It can be ensured that only sharp and/or detailed images of the recognition feature can be captured. The environment of the recognition feature and/or the display device, on which the recognition feature can be displayed, can be unrecognizable. For example, if a patient is within a capture range of the imaging instrument's camera, it can be ensured that the patient is not identifiable from the image of the recognition feature. This may be necessary, for example, due to legal regulations, in particular if the image of the recognition feature is further processed and/or stored centrally, in particular in a cloud. According to some embodiments, at least substantially all image areas of the image are unrecognizable and/or blurred. “Unrecognizable” may mean that at least substantially no details, in particular objective and/or physical details, are recognizable at least to the human eye. Unrecognizable can comprise, for example, blurred, smudged, underexposed and/or overexposed. These features differ from the subsequent blurring of image areas that do not contain the recognition feature. For example, the subsequent application of a filter to these image areas is not comprised by these features. The image data can be generated in such a way that the image areas are already unrecognizable according to the original image data. The recognition unit can be adapted to recognize the recognition feature in an unrecognizable image of the recognition feature.

In addition, the image areas may be unrecognizable due to defocusing of the camera. It is a simple and cost-effective way of ensuring that no recognizable images of patients are taken. In particular, defocusing may mean that the camera is defocused in such a way that, under normal circumstances and with the usual use of the imaging instrument, a recognizable image of a patient would be produced. For example, the camera can be focused on a particularly small and/or a particularly large focal point. Particularly small and/or large is to be seen in connection with a common focal point. For example, the focal point may be three times, four times, five times, ten times and/or twenty times as small and/or as large as a minimum value and/or a maximum value of the focal point during a usual use of the imaging instrument such as for performing a therapeutic and/or diagnostic action. The camera can be defocused in the pairing mode. For example, if the imaging instrument is set to pairing mode, defocusing is set automatically. In this state, the user cannot take recognizable images of the patient.

Furthermore, the recognition feature may be such that it is recognizable in an image of the recognition feature taken in a defocused state of the camera. Advantageously, it is not necessary to ensure that a recognizable image of the recognition feature is captured. Pairing and/or activation can also be carried out using unrecognizable images. This can reduce the technical and/or design demands on the medical system. The medical system can be substantially less complex and at the same time more reliable and/or safer to operate. The recognition feature can, for example, be such that no recognizable image, in particular an image showing representational and/or physical details, is required for recognition. The defocused state can correspond to defocusing.

Furthermore, the recognition feature may comprise a color code. A color code can be such that it is recognizable in an image of the recognition feature taken in the defocused state of the camera. Consequently, no recognizable image of the recognition feature needs to be captured. The color code can comprise a strip code, (different) colored tiles and/or a colored pattern.

Furthermore, the recognition feature may include a temporal color sequence. A color sequence can be such that it is recognizable in an image of the recognition feature taken in the defocused state of the camera. Consequently, no recognizable image of the recognition feature needs to be captured. The color sequence can comprise a successive fade-in of a monochrome representation in different colors. The fade-in may comprise a representation over at least substantially the entire display portion of the display device. A color sequence can, for example, comprise at least two, three, four, five, six and/or seven colors and/or colored representations. An example of a color sequence could be green-red-green, red-blue-yellow-red and/or blue-red-red-blue. The representations can be displayed with a cadence of one representation per second, per half second and/or per quarter second. According to some embodiments, the cadence may correspond to an image refresh rate of the display device.

Furthermore, the recognition feature can comprise a temporal light-dark sequence. A light-dark sequence can be such that it is recognizable in an image of the recognition feature taken in the defocused state of the camera. Consequently, no recognizable image of the recognition feature needs to be captured. The light-dark sequence can comprise a successive fade-in of a monochrome representation, in particular a white and/or a black representation. The representation can be generated in different brightness values. In particular, the temporal light-dark sequence can comprise the fading in of the same representation with different brightness values. A color sequence can, for example, comprise at least two, three, four, five, six and/or seven representations of different brightness. The light-dark sequence can be pulsating. This can mean that there can be a smooth transition between light and dark representations. The time sequence of the brightness value can, for example, be described using a curve function, in particular a sine function. The representations can be displayed with a cadence of one representation per second, per half second and/or per quarter second. According to some embodiments, the cadence may correspond to an image refresh rate of the display device.

In addition, the recognition feature may comprise a barcode. Barcodes have proven to be reliable and favorable recognition features. The barcode can be generated and/or displayed. The barcode can be used to transmit additional information when an image of the barcode is generated and/or analyzed.

The recognition feature can also comprise a QR code. QR codes have proven to be reliable and favorable recognition features. The QR code can be generated and/or displayed. The QR code can be used to transmit additional information when an image of the QR code is generated and/or analyzed.

In addition, the recognition feature can be linked to an instrument identification. If the recognition feature is recognized, the instrument identification can be used to safely and reliably identify a specific imaging instrument. The instrument identification allows at least substantially complete tracking of uses of the imaging instrument and/or functions of the imaging instrument.

Furthermore, the object can comprise a patient file. This may make it possible to trace the data back to a specific patient and/or to trace the use of an imaging instrument in connection with a specific patient. For example, a barcode and/or a QR code can be printed in the patient file, which is clearly assigned to the patient and can be used to identify the patient. When the camera captures an image of the recognition feature that is recognized by the recognition unit, the control unit can enable a sharp, focused image acquisition function and, for example, clearly assign generated image data to the patient. The generated image data and/or information based on the image data can be reliably and securely assigned to an electronic patient file of the patient and stored therein.

The object may also comprise an item of clothing. The identity of the user and/or the patient can be inferred from the item of clothing. For example, a name tag, a barcode, a QR code and/or the like can be attached to the item of clothing and/or integrated into it. If the recognition feature is identified, the user can be inferred and/or it can be recorded which user, in particular when and/or where, has used the imaging instrument. Measures can also be taken, such as those relating to the patient file.

Furthermore, the method of operating a medical system may comprise the step of recognizing the presence of the recognition feature in the image of the recognition feature captured by the camera, the step of generating a recognition signal in accordance with the recognition of the presence of the recognition feature, and the step of activating at least one function of the medical system related to the object in accordance with the recognition signal.

When the object comprises a display device adapted to receive image data and generate a representation for a user, the method of operating a medical system may further comprise the step of displaying the recognition feature on the display device.

The gesture recognition unit can also be arranged on the display device. Advantageously, a compact medical system can be provided that can be set up in a new treatment room in particular as required and/or quickly. The gesture recognition unit can, for example, comprise an attachable and/or connectable module that can be arranged on an existing display device. A medical system can thus be expandable.

In addition, the medical system may comprise a further display device adapted to receive image data and generate a representation for a user, wherein the further display device is paired with the medical imaging instrument, and wherein the gesture recognition unit is adapted to recognize the gesture of the user directed towards the display device and, in accordance with the recognition of the gesture, to establish a pairing between the imaging instrument and the display device in addition to the pairing with the further display device such that representations based on the image data are displayable both on the display device and on the further display device. Advantageously, the display device can be added, wherein the further display device is already paired. The further display device can be, for example, a mobile portable monitor for the imaging instrument on which the user can observe an action. This monitor can be small, which means that it is intended to be used by only one user. To enable other users and/or observers to monitor the action, the display device can be switched on by means of the user's gesture. In this case, the display device can be a large screen in an operating room, for example. At least substantially the same representation can be displayed on both display devices. The representation can be generated centrally and data can be transmitted to the display devices, on the basis of which the display device generates the representation. For example, both display devices can be connected to a central computing unit adapted to generate the representation.

The gesture recognition unit can also be arranged on the further display device. Advantageously, flexibility can be increased and the medical system can be provided as a modular system. Additional computing capacities and/or the like can be added. The gesture recognition unit can be arranged together on the display device and the further display device.

In addition, the imaging instrument and the display device can be paired such that the image data and/or information relating to the representation can be transmitted from the further display device to the display device. Advantageously, interaction between the display devices can be achieved. Not every display device needs to have sufficient computing capacity, in particular graphical computing capacity, to generate the representation. For example, a central display device can be provided which generates the representation and transmits it and/or information and/or data relating to the representation to the other display devices. Display devices can be added in a simple and/or efficient manner. For example, a representation can be duplicated and/or mirrored.

In addition, the gesture recognition unit can comprise at least one gesture recognition module and/or can be extended by connecting further gesture recognition modules. Advantageously, the medical system can be provided in a modular manner and/or be efficiently expandable. For example, the medical system can be quickly and easily adapted to a new working environment, such as a new treatment room. Spatial conditions can be taken into consideration. Furthermore, if the user is partially obscured from the gesture recognition module by another medical device, such as a surgical robot, for example, another gesture recognition module can be added quickly and easily. A gesture recognition module may, for example, comprise a camera, an electromagnetic sensor and/or the like. The gesture recognition module can be adapted to determine a movement of the user and/or the imaging instrument. The gesture recognition unit can be adapted to recognize the gesture in accordance with the movement. According to some embodiments, a plurality of gesture recognition modules may be permanently mounted on a wall of a treatment room. If it turns out that the existing gesture recognition modules are not sufficient to reliably recognize a gesture, an additional gesture recognition module can be added as required.

If the gesture recognition module is a camera, it can be placed on a portable tripod and aligned for gesture recognition.

Furthermore, the medical system may comprise a plurality of display devices each adapted to receive image data and generate a representation for a user, wherein the gesture recognition unit is adapted to recognize a gesture of the user directed to a particular display device of the display devices and to establish a pairing between the imaging instrument and the particular display device of the display devices in accordance with the recognition of the gesture directed to the particular display device of the display devices. Advantageously, the gesture recognition unit can recognize the exact display device to which the gesture is directed, and this display device can be added. The user can therefore decide which of a plurality of display devices he wants to add. It is common for a plurality of display devices to be arranged in treatment rooms. It is therefore advantageous that the gesture recognition unit recognizes the gesture directed at a specific display device. The display devices can be added one after the other.

The gesture recognition unit can also comprise a stereoscopic camera system. Advantageously, a gesture can be spatially detectable and distances can be determined. This improves the reliability and accuracy of the gesture recognition unit. The stereoscopic camera system can be used to generate stereo images. For example, the stereoscopic camera system comprises at least two image acquisition units that generate images of the same object area. A disparity between the images can be used to generate a spatially resolved image, in particular a stereo image. The stereoscopic camera system can be formed by at least two gesture recognition modules, for example. Alternatively or additionally, a gesture recognition module may comprise and/or form the stereoscopic camera system.

Further, the medical system may comprise a microphone adapted to receive an audio signal, wherein the gesture recognition unit is adapted to recognize the audio signal and to prevent or allow pairing between the imaging instrument and the display device in accordance with the recognition of the audio signal. This can improve operating safety and reduce the risk of unintentional pairing. The audio signal may comprise a sound generated by the user. For example, the audio signal may comprise a signal generated by means of the user's vocal cords, in particular a voice command, and/or comprise a sound gesture, such as a clapping, tapping, whistling and/or stamping of the user. In particular, the gesture recognition unit can be adapted for voice recognition. This allows the gesture recognition unit to interpret an audio signal that comprises a voice command. A voice command can comprise a pairing command, for example. By recognizing the command, a pairing mode can be activated in which pairing is possible.

The present disclosure is described below by way of example with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using numerical words, such as first, second, third object, etc., these are used to name and/or assign objects. Accordingly, for example, a first object and a third object may be included, but not a second object. However, a number and/or sequence of objects could also be derived using numerical words.

### BRIEF DESCRIPTION OF DRAWINGS

FIG. 1 shows a schematic representation of a medical system;

FIG. 2 shows a schematic representation of a view of a data transmission adapter;

FIG. 3 shows a schematic representation of a section of the medical system;

FIG. 4 shows a schematic representation of a medical system;

FIG. 5 shows a schematic representation of a medical system;

FIG. 6 shows another schematic representation of the medical system;

FIG. 7 shows a schematic representation of an image of a recognition feature;

FIG. 8 shows a schematic flow chart of a method.

FIG. 9 shows a schematic flow chart of a method.

FIG. 10 shows a schematic flow chart of a method;

FIG. 11 shows a schematic flow chart of a method; and

FIG. 12 shows a schematic flow chart of a method.

### DETAILED DESCRIPTION OF PREFERRED EMBODIMENTS

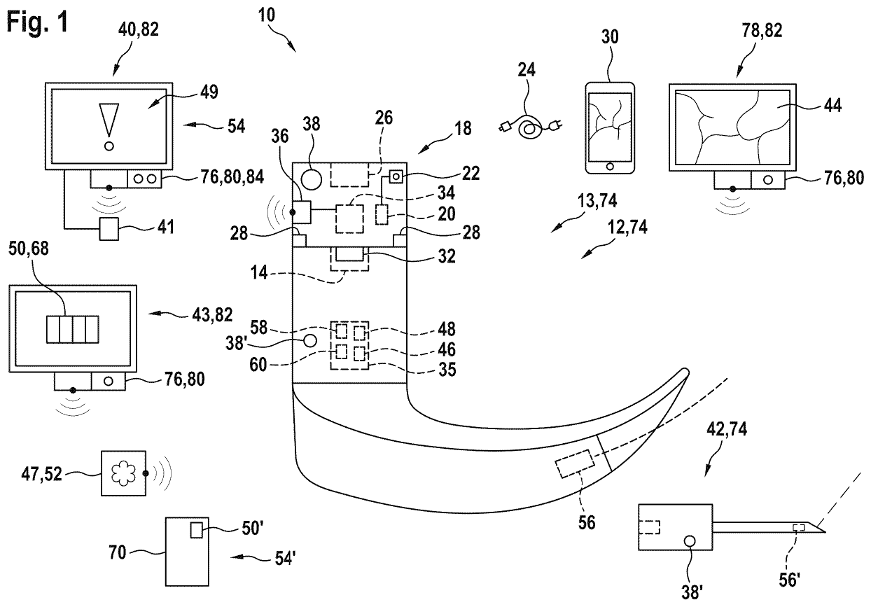

FIG. 1 shows a medical system 10 comprising a plurality of display devices 82, in particular a display device 40, a further display device 43 and a further display device 78, a mobile terminal 30, a data transmission adapter 18, a medical imaging instrument 12, a further medical imaging instrument 42, a gesture recognition unit 76 and a storage unit 52. The display devices 82 can be interchangeable. This means that they can have at least substantially the same features. The assignment of the reference signs can be arbitrary in this respect.

The medical imaging instrument 12 comprises a camera 56 and a plug port 14, to which a data cable (not shown) is typically connected to transmit image data generated by means of camera 56 to at least one of the plurality of display devices 82. Instead of the data cable, the data transmission adapter 18 is connected to the plug port 14. The data transmission adapter 18 is adapted to receive image data via the plug port and to transmit data based on the image data wirelessly. This means that the imaging instrument 12 is adapted for wireless data transmission. Optionally, the data transmission adapter 18 may be connected to the further medical imaging instrument 42 to set it up for wireless data transmission. The imaging instruments 12, 42 are endoscopes 72 in the present case. The imaging instrument 12 is a laryngoscope and the imaging instrument 42 is an endoscope 72 with a rigid shaft. However, the data transmission adapter 18 may in principle be compatible with any imaging instrument.

The imaging instrument 12 and the data transmission adapter 18 form an imaging instrument 13 that can be handled by a user as a single instrument. This means that the imaging instrument 13 can be used as if it had an integrated device for wireless data transmission. The imaging instrument 12 is thus forward compatible, so it can be operated with new display devices that are only set up for wireless data transmission. The data transmission adapter 18 can be used to replace the data cable. In the following, some features of a wireless imaging instrument are described with reference to the imaging instrument 12. In principle, it may be irrelevant whether the imaging instrument 12 or an imaging instrument that is set up for wireless data transmission without a data transmission adapter 18 is meant. In other words, it may not matter how the data is transmitted wirelessly. In particular, the imaging instrument 12 may refer to an imaging instrument that is in a connected state with the data transmission adapter 18, that is, the imaging instrument 13.

Furthermore, image data can be transmitted directly to the mobile terminal 30, in this case a smartphone, using the data transmission adapter 18. This allows the imaging instrument 12 to be used in a highly flexible and/or mobile manner. The smartphone may have appropriate software to operate the imaging instrument 12.

The data transmission adapter 18 comprises a connection element 32, a sealing device 28, a charging interface 22, an energy storage 20, a control unit 34, a data interface 36, an operating element 38 and a plug port 26. The connection element 32 is formed like a plug of the data cable, which would normally be connected to the plug port 14 of the imaging instrument 12. The connection element 32 has a plug-like design. The data transmission adapter 18 receives image data from the imaging instrument 12 via the connection element 32 and forwards it to the control unit 34. The control unit 34 is used to prepare the image data. This can comprise cleansing, compressing, encrypting, packing into individual packages and/or the like. In particular, the image data is prepared in such a way that it can be transmitted wirelessly. Data based on the image data, in particular prepared image data, is forwarded to the data interface 36. By means of the data interface 36, this data can be transmitted wirelessly to a receiving device. For example, according to FIG. 1, the data is transmitted to the display device 78 and a representation 44 based on the data, in particular image data, is generated by the display device 78. Furthermore, the data transmission adapter 18 can receive at least one control signal wirelessly via the data interface 36, prepare it by means of the control unit 34 and transmit it to the imaging instrument 12 via the connection element 32. The user can, for example, generate an image acquisition command at an input device 41 arranged at the display device 40, which is transmitted to the data transmission adapter 18. This allows the user to activate a function of the camera 56.

By means of the energy storage 20, the imaging instrument 12 is supplied with energy, in particular electrical energy, via the plug port 14 during operation. In the present case, the energy storage 20 is a rechargeable energy storage 20, in particular an accumulator, which is integrated into the data transmission adapter 18. In particular, the energy storage 20 is integrated such that a mounting chamber for the energy storage 20 can be closed off from the environment in a gas-tight and/or fluid-tight manner. In order to be able to recharge the energy storage 20, the data transmission adapter 18 comprises the charging interface 22. The charging interface 22 is adapted for inductive energy transmission and is designed for inductive charging by means of an inductive charging station (not shown). In addition, the charging interface 22 comprises a socket for a plug of a charging cable 24. The charging cable 24 can optionally be detachably connected to the data transmission adapter 18 in order to charge it.

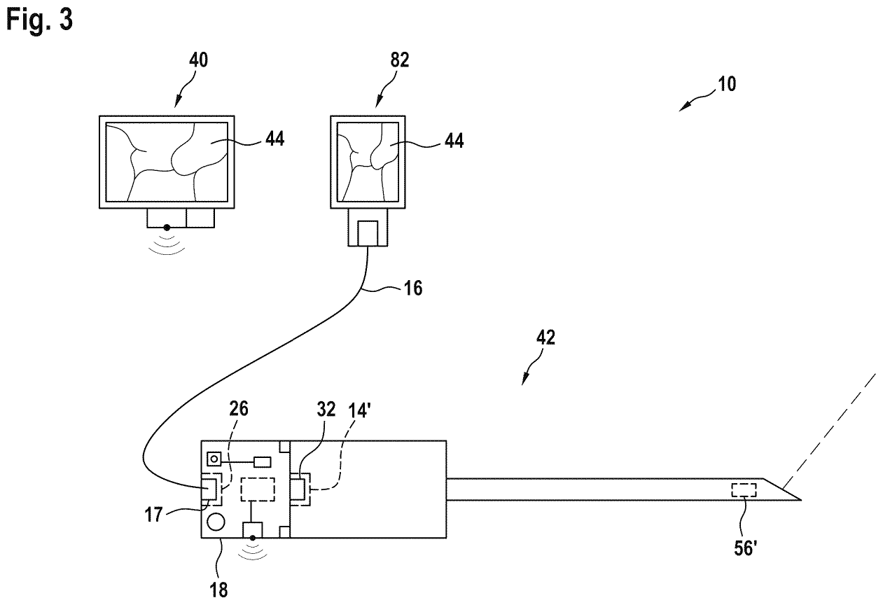

The plug port 26 of the data transmission adapter 18 is substantially the same as the plug port 14 of the imaging instrument. This means that the plug port 26 and the plug port 14 have substantially the same design. The data cable can be connected to plug port 26, which can be connected to plug port 14. This allows the data transmission adapter 18 to be operated simultaneously with a wired display device and a wireless display device (see FIG. 3).

An operating element 38 can also be used, for example, to activate and/or control a function of the imaging instrument 12. In addition, the operating element 38 can be used to set the data transmission adapter 18 to a pairing mode for establishing a pairing with a display device and/or to confirm a pairing. This will be discussed further below.

The sealing device 28 is provided to allow the data transmission adapter 18 to be disinfected and/or autoclaved together with the imaging instrument 12 in a state connected to the imaging instrument 12. In the connected and/or mated state, the sealing device 28 is used to seal the plug port 14 in a fluid-tight and/or gas-tight manner with respect to the environment. The sealing device 28 is provided as a deformable rubber coating along the entire circumference of a contact surface to the imaging instrument 12 of the data transmission adapter 18. When the data transmission adapter 18 is connected, the rubber coating is subjected to a force and thus deformed so that the plug port is sealed off from the environment. Alternatively or additionally, for example, an O-ring can also be provided on the connection element 32.

According to the schematic representation of FIG. 1, the further display device 78 is paired with the medical imaging instrument 12 and data based on the image data is transmitted to the display device 78. The display device is adapted to generate a representation 44 for the user based on the data. This allows the user to observe an imaged object area during use of the imaging instrument 12 on the display device 78. Further display devices 40, 43 may optionally be additionally paired with the imaging instrument 12 and the representation may be generated by and/or displayed on them. Thus, at least substantially the same representation 44 may be displayed on different display devices 82.

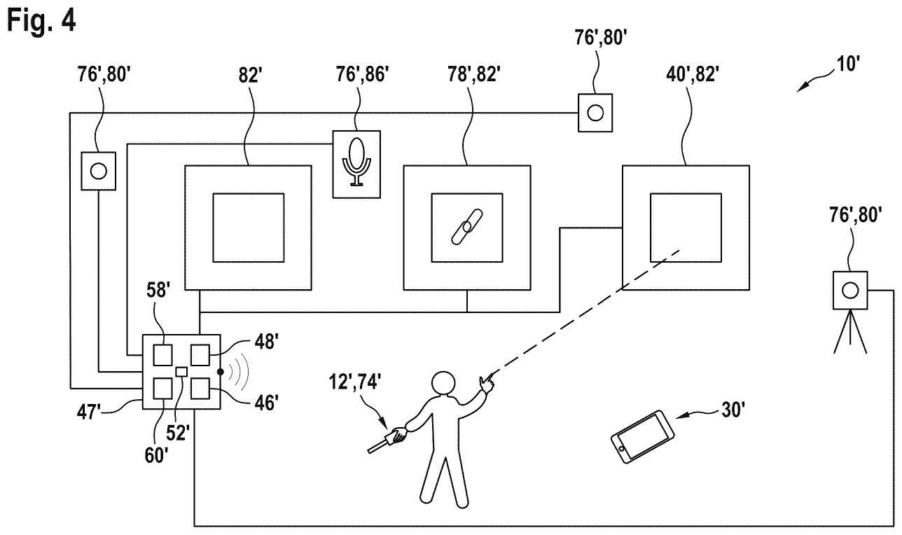

Various processes and methods may be provided for pairing at least one of the display devices 40, 43. For example, the pairing mode can be initiated by an actuation of the operating element 38 of the data transmission adapter 18. According to another of the methods, a gesture recognition unit 76 is provided. It is described again in more detail in connection with FIG. 4. The gesture recognition unit 76 comprises a plurality of gesture recognition modules 80, each arranged on one of the display devices 82, 40, 43, 78. The gesture recognition modules 80 each comprise a camera arranged and adapted to image the user using the imaging instrument 12. The gesture recognition module 80 on the display device 40 comprises a stereoscopic camera system 84. Alternatively or additionally, the gesture recognition modules 80 may together form the stereoscopic camera system 84. The gesture recognition unit 76 is adapted to recognize a gesture of the user directed towards the corresponding display device 40, with which the user wishes to pair the imaging instrument 12, and to establish a pairing between the imaging instrument 12 and the display device 40 in accordance with the recognition of the gesture. For this purpose, the user can, for example, point with one hand in the direction of the display device 40 to be paired. This gesture is recognized by the gesture recognition unit 76, in particular by means of spatial resolution. The further display device 43 can also be paired in the same way. Furthermore, the gesture recognition unit 76 can be expanded by adding further gesture recognition modules 80. These can also be arranged on further display devices and/or be stand-alone gesture recognition modules.

According to the embodiment example in FIG. 1, the pairing is carried out using a two-step method. In the first step, the pairing is initiated using the gesture recognition unit 76, or the imaging instrument 12 and/or the data transmission adapter 18 is set to a pairing mode. Alternatively or additionally, the pairing is initiated by actuating the operating element 38 of the data transmission adapter 18, or the imaging instrument 12 and/or the data transmission adapter 18 is set to the pairing mode. In a second step, it is checked whether the initiated pairing is an intended pairing, i.e. whether the imaging instrument 12 is actually to be paired with the display device 40. For this purpose, the imaging instrument 12 comprises a pairing unit 46 and a security unit 48. By means of the coupling unit 46 and the security unit 48, any coupling can in principle be checked, regardless of whether it was initiated by means of the gesture recognition unit 76.

The pairing unit 46 is adapted to establish the pairing of the imaging instrument 12 and the display device 40, wherein in a paired state the image data can be transmitted from the imaging instrument 12 to the display device 40. The security unit 48 is adapted to recognize a confirmation of the pairing by the user, wherein the security unit 48 is adapted to prevent the transmission of the image data from the imaging instrument 12 to the display device 40 in the absence of the confirmation of the pairing by the user. If there is no confirmation from the user, the pairing of the imaging instrument 12 and the display device 40 can be released.

When the presence of a pairing attempt is detected, in particular by means of the pairing unit 46, the security unit 48 generates a request 49 to the user to confirm the pairing. According to the embodiment shown, the display device 40 displays the request 49 to the user. This may comprise fading in a request display via a representation of the display device 40. During the pairing process, the imaging instrument 12 and the display device 40 are already paired in such a way that the request 49 can be transmitted to the display device 40. However, at least image data and/or data based on the image data are not transmitted. Alternatively or additionally, the imaging instrument 12 may comprise at least one LED (not shown) and/or a speaker (not shown) by means of which the user may be requested to confirm the pairing. For example, the LED can flash in a particular color, such as red, to prompt the user to confirm. In a coupled state, the LED can light up in a different color and/or color mixture, e.g. in white. The loudspeaker can be used to emit a sound recording of a spoken request and/or a signal tone, for example.

To confirm the pairing, the user actuates the operating element 38 of the data transmission adapter 18 and/or a control element 38′ of the imaging instrument 12. Confirming comprises actuating the operating element 38, 38′ over a predetermined minimum confirmation time period, wherein the confirmation time period is preferentially at least three seconds long and preferably at least five seconds long. Once the pairing is confirmed, the security unit 48 is adapted to allow transmission of the image data from the imaging instrument 12 to the display device 40. In this case, the user has confirmed that the correct display device is paired with the correct imaging instrument 12.

Alternatively or additionally, confirming the pairing comprises capturing an image of a recognition feature 50 using the imaging instrument 12. In such a case, the display device 40 generates a recognition feature by means of which the display device 40 can be identified. This will be discussed in more detail below. The representation of the recognition feature can be generated after the request 49 is displayed and/or can be displayed together with the request.

Thus, if the user wants to pair the imaging instrument 12 with the display device 40, the user can select the display device 40 by means of a gesture. A request 49 to confirm the pairing is then displayed on the display device 40. In addition, a recognition feature is displayed on the display device 40, from which the user captures an image using the imaging instrument 12 to confirm pairing. Alternatively, the user can initiate and/or confirm pairing by actuating the operating element 38.

Alternatively or additionally, pairing can be prevented and/or permitted by applying an assignment rule. For this purpose, a first device identification is assigned to the imaging instrument 12 and a second device identification is assigned to the display device 40. In the pairing unit 46, the assignment rule is also stored, which defines combinations of different device identifications as permissible or impermissible. Alternatively or additionally, the assignment rule may be stored on a storage unit 52 of a central computing unit 47. The central computing unit 47 may form the pairing unit 46 together with the imaging instrument 12. The pairing unit 46 is adapted to compare the first device identification with the second device identification using the association rule, and to establish or prevent pairing of the imaging instrument 12 and the display device 40 in accordance with the application of the association rule. If the gesture is used to select one of the display devices to establish pairing, the assignment rule can be used to check whether the desired pairing is to be established or whether pairing is to be prevented. For this purpose, the assignment rule may comprise preset device identifications of imaging instruments and display devices present in a particular operating room and/or the like. If a device identification is recognized that can be assigned to these present devices, pairing is permitted. Otherwise it will be prevented.

Further, the security unit 48 is adapted to determine a user authorization of the user, compare the user authorization with a predetermined required authorization, and prevent transmission of the image data from the imaging instrument 12 to the display device 40 if the user authorization does not reach the predetermined required authorization. The user authorization can also be stored on the central computing unit 47, for example. The central computing unit 47 is intended, for example, to manage the apparatuses in a hospital. By storing the user authorization, a desired pairing of apparatus can be set in advance and/or activated by payment. The user can select a desired pairing in the software and pay an amount to obtain user authorization. This allows a billing method to be implemented for the use of imaging instruments and display devices in which each use is billed individually.

The storage unit 52 also stores information about the pairing of the imaging instrument 12 with the display device 40 and/or the confirmation of the pairing by the user. This makes it possible, for example, to determine how long and how often which apparatuses have been used. Information about unsuccessful pairing attempts can also be stored. In particular, the storage unit 52 may comprise a cloud-based storage unit. This allows decentralized access to the relevant information.

The imaging instrument 12 further comprises a recognition unit 58 adapted to detect the presence of a recognition feature 50 in an image of the recognition feature 50 captured by the camera 56 and to generate a recognition signal as a function of the recognition feature 50, and a control unit 60 adapted to process the recognition signal and to activate at least one function of the medical imaging instrument 12 in accordance with the recognition signal.

The further display device 43 is adapted to display the recognition feature 50. The recognition feature 50 comprises a color code 68 and is such that it is recognizable in an image of the recognition feature 50 taken in a defocused state of the camera 56.

According to the case described above, the function related to the object 54 is the pairing of the imaging instrument 12 with the display device 40. The object 54, corresponding to the display device 40, is identifiable by the recognition feature 50 when the recognition feature 50 is displayed to confirm pairing. In this case, the recognition feature 50 may be associated with the instrument identification of the display device 40. Thus, when the recognition feature 50 is displayed, the user may capture an image of the recognition feature 50 to perform the pairing of the imaging instrument 12 to be paired with the display device.

According to the embodiment described below, the function is an activation of a sharp, focused image acquisition by means of the camera 56. Before activation, the camera 56 can only capture images in such a manner that at least substantially the entire image area is unrecognizable. This is because the camera 56 is defocused, for example, and, in particular, is focused on a very close or very distant point. Activation can be initiated by actuating the operating element 38, 38′. This causes the recognition feature 50 to be displayed on the further display device 43. The user can then capture an image of the recognition feature 50 using the defocused camera 56, wherein, in particular, no sharp images of a patient are captured prior to activation of the sharp image acquisition. When the recognition feature 50 is recognized, the sharp image acquisition is activated and a diagnostic action can be performed by the imaging instrument 12.

The object 54′ can also comprise a patient file 70. The patient file 70 has the recognition feature 50′, in particular a QR code 66 and/or barcode 64, by means of which the patient file 70 and/or the patient to be treated can be identified. For example, when the user generates an image of the recognition feature 50′ by means of the camera 56 of the imaging instrument 12, data, in particular image data, generated in response can be associated with the patient and/or the patient file 70 by means of the control unit 60, which is a function related to the object 54′. In particular, the patient file 70 may also comprise an electronic patient file and the corresponding recognition feature 50′ may be displayed on one of the display devices 82. In addition, a name tag (not shown) on which a recognition feature, for example a QR code, is arranged can be attached to a patient's bedside. Before using the imaging instrument 12 to perform a therapeutic and/or diagnostic action, the user can create an image of the QR code. Subsequently performed actions can then be assigned to the patient and, for example, information can be stored in, in particular in the cloud-based, storage unit 52.

According to the embodiment example of FIG. 1, the imaging instrument 12 comprises the recognition unit 58, the control unit 60, the pairing unit 46 and the security unit 48. These may be implemented together on a computing unit 35 of the imaging instrument 12. Such an imaging instrument 12 is particularly suitable for mobile applications, for example in conjunction with the mobile terminal 30. The mobile terminal can have at least some of the other features described. Alternatively or additionally, these units 46, 48, 58, 60 may be located at least partially external to the imaging instrument 12. For example, the further imaging instrument 42 does not comprise any of these units. If the further imaging instrument 42 is set up for wireless data transmission by means of the data transmission adapter 18, these units may be implemented on an external computing unit (see, for example, FIG. 4). Furthermore, it is understood that the individual features described above are optional and pairing can also be carried out without the recognition unit 58 and/or the gesture recognition unit 76, for example.

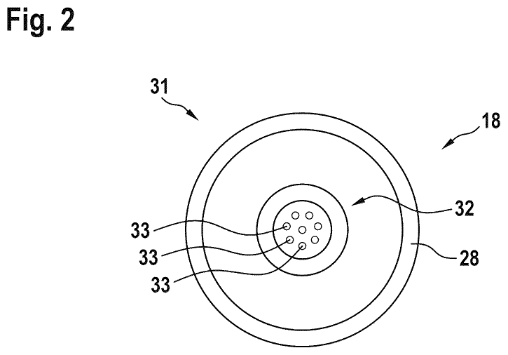

FIG. 2 shows a schematic representation of a view of the data transmission adapter 18. The contact surface 31 of the data transmission adapter 18 is shown, which, in a state connected to an imaging instrument, is at least partially in contact with a proximal end surface of the imaging instrument. The sealing device 28 in the form of the rubber coating is arranged along the circumference of the contact surface 31. In the center of the contact surface 31, the connection element 32 extends away from the contact surface 31. The connection element 32 is designed like a plug for a standard data cable used with the corresponding imaging instrument. By way of example, the connection element 32 is shown with eight pins 33, each of which is plugged into a corresponding socket of the plug port 14 in order to connect the data transmission adapter 18. As an alternative or in addition to the pins, spring contact pins, for example pogo pins, and corresponding contact surfaces can also be provided. The contour of the contact surface can be designed such that it corresponds to a contour of the proximal end surface of an imaging instrument.

FIG. 3 shows a schematic representation of a section of the medical system 10 of FIG. 1. The data transmission adapter 18 is connected to the further medical imaging instrument 42. For this purpose, the connection element 32 of the data transmission adapter is plugged into the plug port 14′ of the imaging instrument 42. The data transmission adapter 18 is coupled to the display device 40 for wireless data transmission. This generates the representation 44 of an object area which is imaged by the camera 56′ of the imaging instrument 42. A further display device 82 is also shown. This display device 82 is an old wired display device that was commonly used with the imaging instrument 42. However, the imaging instrument 42 has been set up for wireless data transmission by means of the data transmission adapter 18. In addition, the data transmission adapter 18 is connected to the old display device 82 by means of the data cable 16. The data cable 16, which comprises a plug 17, is connected to the display device 82 and by means of the plug 17 to the plug port 26 of the data transmission adapter 18. The plug 17 is designed in the same way as the connection element 32. The plug 17 can therefore also be plugged into the plug port 14′. The plug port 26 is also designed in the same way as the plug port 14′. Thus, at least substantially the same representation 44 may be displayed on the display device 82. However, a resolution of the representation 44 and/or a format of the representation 44 may be different. At least the representations 44 of each of the display devices 40, 82 are based on the same image data.

FIG. 4 shows a schematic representation of a further embodiment of a medical system 10′. The medical system has a lot in common with system 10. The reference signs are provided with quotation marks in order to emphasize a similarity to the medical system 10. The main focus is on differences. The function of the individual units can be the same.

In contrast to FIG. 1, the pairing unit 46′, the security unit 48′, the recognition unit 58′ and the control unit 60′ are arranged in a central computing unit 47′ or implemented thereon. The computing unit 57′ also comprises a storage unit 52′. The computing unit 57′ is a computer in an operating room and/or alternatively a server of a hospital. The computer and the server can also complement each other, and the units can draw on the computing power of both.

The computing unit 47′ is connected to display devices 82′, 40′ 78′. In addition, the computing unit 47′ is set up for wireless data transmission. For example, this allows control commands and/or signals from the units 46′, 48′ 58′ 60′ to be transmitted to an imaging instrument 12′. The imaging instrument 12′ is an endoscope 74 that has an integrated data interface. Alternatively, the imaging instrument 12′ can be the imaging instrument 12 of FIG. 1 and can be set up for wireless data transmission by means of the data transmission adapter 18.

The display devices 82′ are interconnected and set up to exchange data. The imaging instrument 12′ is paired with the display device 78′. When the display device 40′ is also paired with the imaging instrument 12′, image data and/or information relating to a representation is transmitted from the further display device 78′ to the display device 40′. For example, a representation is duplicated on the other paired display device 40. Furthermore, the information relating to the representation can also be transmitted to the mobile terminal 30′.

Furthermore, the gesture recognition unit 76′ differs from that of FIG. 1. The gesture recognition unit 76′ comprises a plurality of independent gesture recognition modules 80′, each comprising a camera. Together, the gesture recognition modules 80′ form a stereoscopic camera system. This means that the user can be imaged from several directions and a gesture by the user can be interpreted and/or recognized spatially. When the user points to the display device 40′, as shown, this gesture is recognized by the gesture recognition unit 76′ and a pairing between the imaging instrument 12′ and the display device 40′ is established in accordance with the recognition of the gesture. Alternatively or additionally, the user can point to the display device 40′ with the imaging instrument 12′. Again, alternatively or additionally, optical markers (not shown) may be arranged on the imaging instrument 12′, by means of which recognition of the gesture is simplified. Again alternatively or additionally, the gesture recognition unit 76′ may comprise an electromagnetic measuring device (not shown) and/or the imaging instrument 12′ may comprise electromagnetic markers (not shown) and the gesture may be recognized by detecting the position of the electromagnetic markers. The gesture recognition unit 76′ also comprises a microphone 86′, which is adapted to receive an audio signal. The gesture recognition unit 76′ is adapted to recognize the audio signal and to prevent or allow pairing between the imaging instrument 12′ and the display device 40′ in accordance with the recognition of the audio signal. Furthermore, a pairing mode can be activated according to the recognition of the audio signal. For example, the user can generate an audio signal to confirm pairing. For example, if pairing is performed, a request to confirm pairing can be generated by the security unit 60′ and the user can confirm pairing by means of the audio signal. The audio signal can be a voice command and/or a deliberately generated signal such as a clap. In the same way, the further of the display devices 82′ can be coupled.

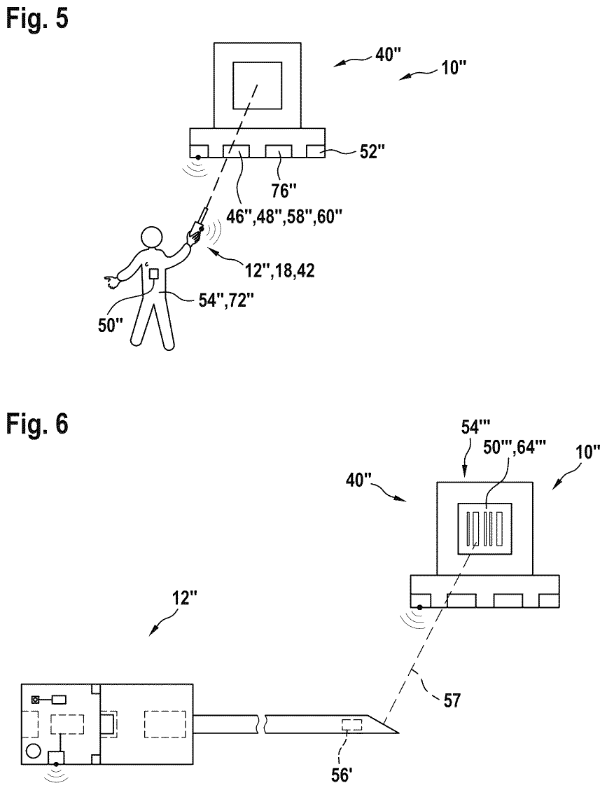

FIG. 5 shows a schematic representation of a further embodiment of a medical system 10″. Due to the many similarities to the medical systems 10, 10′, the differences are primarily discussed. The storage unit 52″, the gesture recognition unit 76″, the pairing unit 56″, the security 48″, the recognition unit 5′″ and the control unit 60″ are arranged on a display device 40″. A user holds a medical imaging instrument 12″ towards the display device 40″, which corresponds to a gesture for pairing. In this way, the user wants to put the imaging instrument 12″ into a pairing mode. This gesture is recognized by the gesture recognition unit 76″. The imaging instrument 12″ is the imaging instrument 42 with the data transmission adapter 18 connected thereto. Alternatively, the imaging instrument 12″ can also be another imaging instrument that is set up for wireless data transmission. In addition, the user wears an item of clothing 72″ on which a recognition feature 50″ is arranged. The item of clothing 72″ may be an object 54″ and the item of clothing 72″ and/or the user may be identifiable from an image of the recognition feature 50″. The detection feature 50′ is linked to an identification of the user. By capturing the image of the recognition feature 50″, it can be determined that the user is using the imaging instrument 12″ and subsequently captured image data can be linked to the user. Furthermore, user authorization to use the imaging instrument 12″ can be checked. Information about the user, the recognition feature 50″ and/or the like is stored on the storage unit 52″.

FIG. 6 shows a further schematic representation of the medical system 10″. An object 54′″ comprises the display device 40″, on which a recognition feature 50′″ in the form of a barcode 64′″ is displayed. Alternatively or additionally, the recognition feature 50′″ may comprise a temporal color sequence and/or a temporal light-dark sequence. The camera 56′ of the imaging instrument 12″ defines a viewing direction 57. To activate a function of the medical system 10″ related to the object 54′″, in the present case the activation of a sharp, focused/focusable image acquisition by means of the camera 56′, the user holds the imaging instrument 12″ such that the recognition feature 50′″ is arranged approximately in the viewing direction 57 of the imaging instrument 12″. In this state, the camera is 56′ defocused and it is not possible to take a sharp picture. In particular, no representation based on image data is shown on a display device. This state can also be referred to as recognition mode. The imaging instrument 12′″ can be set to this mode by actuating the operating element 38. Alternatively or additionally, the imaging instrument 12″ can always be in the recognition mode after being switched on. The user therefore has no feedback as to whether he is holding the imaging instrument 12″ correctly. However, the imaging instrument 12″ conveniently has a focus on a very distant point so that a large image area can be captured. As soon as the image of the recognition feature is captured, the user receives feedback, for example, by displaying a recognition message in the display device 40″ after the recognition signal has been generated. The user moves the imaging instrument 12″ slightly with respect to the display device 40″ until the recognition message is displayed. Once the presence of the recognition feature 50′″ has been recognized, the sharp image acquisition is activated and a patient can be examined.

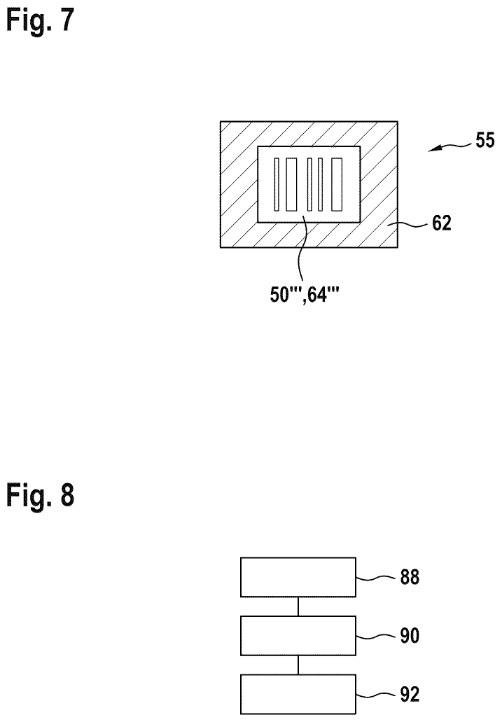

In FIG. 7, a schematic representation of the image 55 of the recognition feature 50′″, in particular the barcode 64′″, is shown. In the recognition mode, the medical imaging instrument 12″ has captured the image 55 of the recognition feature 50′″ of the object 54′″ and/or generated associated image data such that at least the image area 62 that does not contain the recognition feature 50′″ is unrecognizable. In particular, the image area 62 is unrecognizable due to the defocusing of the camera 56′.

FIG. 8 shows a schematic flowchart of a method for retrofitting a medical imaging instrument 12, 42, in particular by means of a medical system 10, 10′, 10″ according to the disclosure and/or by means of a data transmission adapter 18 according to the disclosure. The method comprises a step 88 of providing a medical imaging instrument 12, 42 adapted to generate image data and comprising a plug port 14 for connecting a data cable 16, a step 90 of providing a data transmission adapter 18 detachably connectable to the plug port 14 and adapted to receive and wirelessly transmit the image data via the plug port 14, and a step 92 of connecting the data transmission adapter 18 to the plug port 14 of the imaging instrument 12, 42.

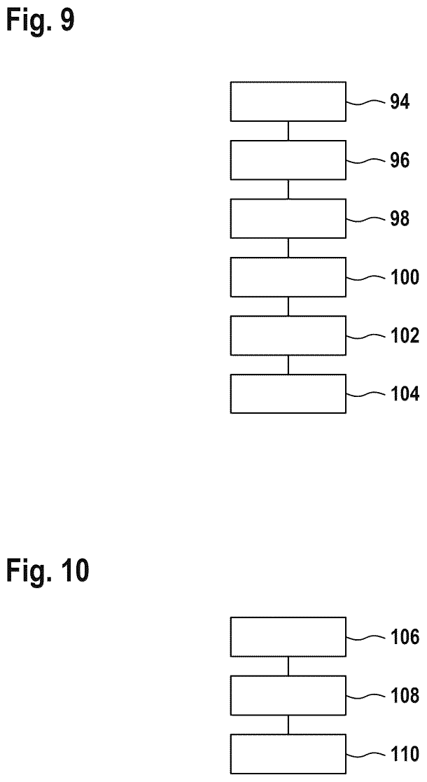

FIG. 9 shows a schematic flowchart of a method for transmitting image data, in particular by means of a medical system 10, 10′, 10″ according to the disclosure and/or by means of a data transmission adapter 18 according to the disclosure. The method comprises a step 94 of providing a medical imaging instrument 12 adapted to generate image data and comprising a plug port 14 for connecting a data cable 16, a step 96 of providing a data transmission adapter 18 detachably connectable to the plug port 14 and adapted to receive and wirelessly transmit the image data via the plug port 14, a step 98 of connecting the data transmission adapter 18 to the plug port 14 of the imaging instrument 12, a step 100 of generating image data by means of the imaging instrument 12, a step 102 of transmitting the image data to the data transmission adapter 18 via the plug port 14, and a step 104 of transmitting the image data wirelessly by means of the data transmission adapter 18.

FIG. 10 shows a schematic flowchart of a method for connecting a medical imaging instrument 12 to a display device 40, in particular performed by means of a medical system 10, 10′, 10″ according to the disclosure and/or a medical imaging instrument 12 according to the disclosure, wherein the medical imaging instrument 12 is adapted to generate image data, and wherein the display device 40 is adapted to receive image data and to generate a representation 44 for a user. The method comprises a step 106 of establishing pairing of the imaging instrument 12 and the display device 40, wherein, in a paired state, the image data is transmittable from the imaging instrument 12 to the display device 40, a step 108 of recognizing a confirmation of the pairing by the user, and a step 110 of preventing transmission of the image data from the imaging instrument 12 to the display device 40 in the absence of the confirmation of the pairing by the user.

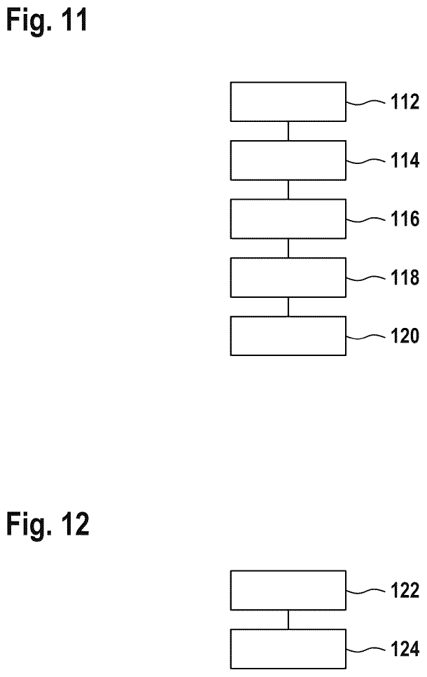

FIG. 11 shows a schematic flowchart of a method with a medical system 10, 10′, 10″ according to the disclosure, wherein the medical system 10, 10′, 10″ comprises an object 54 having a recognition feature 50 and being identifiable by means of the recognition feature 50, and a medical imaging instrument 12 having a camera 56 by means of which images can be captured and image data can be generated, wherein an image of the recognition feature 50 of the object 54 can be captured by means of the camera 56. In addition, the object 54 may comprise a display device 40 adapted to receive image data and generate a representation for a user. The method comprises a step 112 of capturing an image of the recognition feature 50 of the object 54 by the camera 56, a step 114 of recognizing the presence of the recognition feature 50 in the image of the recognition feature 50 captured by the camera 56, a step 116 of generating a recognition signal in accordance with the recognition of the presence of the recognition feature 50, and a step 118 of activating at least one function of the medical system 10 related to the object 54 in accordance with the recognition signal. In addition, the method may comprise a step 120 of displaying the recognition feature 50 on the display device 40.

FIG. 12 shows a schematic flowchart of a method for pairing a medical imaging instrument 12 with a display device 40, in particular with a medical system 10, 10′, 10″ according to the disclosure, wherein the medical imaging instrument 12 is adapted to generate image data and to transmit the image data wirelessly, and wherein the display device 40 is adapted to receive image data and to generate a representation 44 for a user. The method comprises a step 122 of recognizing a gesture of the user directed to the display device 40, and a step of establishing a pairing between the imaging instrument 12 and the display device 40 in accordance with the recognition of the gesture.

The disclosure relates, inter alia, to the following aspects:

Aspect 1. A medical system (10) comprising:
a medical imaging instrument (12) adapted to generate image data and comprising a plug port (14) for connecting a data cable (16); and
a data transmission adapter (18) detachably connectable to the plug port (14) and adapted to receive the image data via the plug port (14) and to wirelessly transmit data based on the image data.

Aspect 2. The medical system (10) according to aspect 1,
wherein the data transmission adapter (18) comprises an energy storage (20) adapted to provide energy to the medical imaging instrument (12) during operation via the plug port (14).

Aspect 3. The medical system (10) according to aspect 2,
wherein the data transmission adapter (18) comprises a charging interface (22) by means of which the energy storage (20) can be charged.

Aspect 4. The medical system (10) according to aspect 3,
wherein the charging interface (22) is adapted for inductive energy transmission.

Aspect 5. The medical system (10) according to aspect 3 or 4,
wherein the charging interface (22) is adapted for detachably connecting a charging cable (24).

Aspect 6. The medical system (10) according to any of the preceding aspects,
wherein the data transmission adapter (18) comprises a plug port (26) for connecting a data cable (16).

Aspect 7. The medical system (10) according to aspect 6,
wherein the plug port (26) of the data transmission adapter (18) corresponds in its design to a design of the plug port (14) of the imaging instrument (12).

Aspect 8. The medical system (10) according to any of the preceding aspects,
wherein the data transmission adapter (18) is adapted to be disinfected and/or autoclaved.

Aspect 9. The medical system (10) according to any of the preceding aspects,
adapted to be at least partially disinfected and/or autoclaved in a state in which the data transmission adapter (18) is connected to the medical imaging instrument (12).

Aspect 10. The medical system (10) according to any of the preceding aspects,
wherein the data transmission adapter (18) comprises a sealing device (28) which is adapted to seal the plug port (14) of the imaging instrument (12) from an environment, in particular in a fluid-tight and/or gas-tight manner, in a state in which the data transmission adapter (18) is connected to the medical imaging instrument (12).

Aspect 11. The medical system (10) according to any of the preceding aspects,
wherein the data transmission adapter (18) is adapted to prepare the image data prior to wireless transmission.

Aspect 12. The medical system (10) according to any of the preceding aspects,
wherein the data transmission adapter (18) is adapted to transmit the image data directly to a mobile terminal (30) wirelessly.

Aspect 13. The medical system (10) according to any of the preceding aspects,
wherein the data transmission adapter (18) is adapted to receive at least one control signal wirelessly and to transmit the control signal to the imaging instrument (12) via the plug port (14).

Aspect 14. The medical system (10) according to any of the preceding aspects,
wherein, in a state in which the data transmission adapter (18) is connected to the medical imaging instrument (12), the data transmission adapter (18) and the imaging instrument (12) together form an imaging instrument (13) which can be handled by a user as a single instrument.

Aspect 15. The medical system (10) according to any of the preceding aspects,
wherein the data transmission adapter (18) comprises:
a connection element (32) which can be detachably connected to the plug port (14);
a control unit (34) connected to the connection element (32) and adapted to receive the image data from the connection element (32); and
a data interface (36) which is connected to the control unit (34) and which is adapted to receive data from the control unit (34) and to transmit data wirelessly.

Aspect 16. The medical system (10) according to any of the preceding aspects,
wherein the data transmission adapter (18) comprises an operating element (38), by means of which the data transmission adapter (18) can be set to a pairing mode for establishing a pairing with a display device (40).

Aspect 17. The medical system (10) according to any of the preceding aspects,
further comprising a further medical imaging instrument (42), wherein the data transmission adapter (18) is selectively connectable to the imaging instrument (12) or the further imaging instrument (42).

Aspect 18. A data transmission adapter (18) for a medical system (10) according to any of the preceding aspects.

Aspect 19. The data transmission adapter (18), in particular for a medical system (10) according to any of aspects 1 to 17, comprising:
a connection element (32) which is detachably connectable to a plug port (14) of a medical imaging instrument;
a control unit (34) connected to the connection element (32) and adapted to receive image data from the connection element (32); and
a data interface (36) which is connected to the control unit (34) and which is adapted to receive data from the control unit (34) and to transmit data wirelessly.

Aspect 20. A method for retrofitting a medical imaging instrument (12, 42), in particular by means of a medical system (10) according to any of aspects 1 to 17 and/or by means of a data transmission adapter (18) according to aspect 18 or 19, comprising:
providing a medical imaging instrument (12, 42) adapted to generate image data and comprising a plug port (14) for connecting a data cable (16);
providing a data transmission adapter (18) detachably connectable to the plug port (14) and adapted to receive and wirelessly transmit the image data via the plug port (14); and
connecting the data transmission adapter (18) to the plug port (14) of the imaging instrument (12, 42).

Aspect 21. A method for transmitting image data, in particular by means of a medical system (10) according to any of aspects 1 to 17 and/or by means of a data transmission adapter (18) according to aspect 18 or 19, comprising:
providing a medical imaging instrument (12) adapted to generate image data and comprising a plug port (14) for connecting a data cable (16);
providing a data transmission adapter (18) detachably connectable to the plug port (14) and adapted to receive and wirelessly transmit the image data via the plug port (14);
connecting the data transmission adapter (18) to the plug port (14) of the imaging instrument (12);
generating image data using the imaging instrument (12);
transmitting the image data via the plug port (14) to the data transmission adapter (18); and
transmitting the image data wirelessly using the data transmission adapter (18).

Aspect 22. The method of operating a medical system (10) according to any of aspects 1 to 17 and/or a medical imaging instrument (12) according to aspect 18 or 19.

Aspect 23. A medical system (10) comprising:
a medical imaging instrument (12) adapted to generate image data;
a display device (40) adapted to receive image data and generate a representation (44) for a user;
a pairing unit (46) adapted to establish a pairing of the imaging instrument (12) and the display device (40), wherein, in a paired state, the image data is transmittable from the imaging instrument (12) to the display device (40); and
a security unit (48) adapted to recognize a confirmation of the pairing by the user, wherein the security unit (48) is adapted to prevent the transmission of the image data from the imaging instrument (12) to the display device (40) in the absence of the confirmation of the pairing by the user.

Aspect 24. The medical system (10) according to aspect 23,
wherein preventing the transmission of the image data comprises releasing the pairing of the imaging instrument (12) and the display device (40).

Aspect 25. The medical system (10) according to any of the preceding aspects, in particular according to any of aspects 23 to 24,
wherein the security unit (48) is adapted to generate a request (49) to the user for confirmation of the pairing.

Aspect 26. The medical system (10) according to aspect 25,
wherein the display device (40) is adapted to display the request (49) to the user.

Aspect 27. The medical system (10) according to any of the preceding aspects, in particular according to any of aspects 23 to 26,
wherein the medical imaging instrument (12) comprises an operating element (38), and
wherein the confirmation of the pairing comprises an actuation of the operating element (38).

Aspect 28. The medical system (10) according to aspect 27,
wherein the confirmation of the pairing comprises an actuation of the operating element (38) over a predetermined minimum confirmation time period, wherein the confirmation time period is preferentially at least three seconds long and preferably at least five seconds long.

Aspect 29. The medical system (10) according to any of the preceding aspects, in particular according to any of aspects 23 to 28,
wherein the security unit (48) is adapted to allow transmission of the image data from the imaging instrument (12) to the display device (40) when the confirmation of the pairing is present.

Aspect 30. The medical system (10) according to any of the preceding aspects, in particular according to any of aspects 23 to 29,
wherein the display device (40) has a recognition feature (50) and is identifiable by means of the recognition feature (50),
wherein the confirmation of the pairing comprises capturing an image of the recognition feature (50) by means of the imaging instrument (12).

Aspect 31. The medical system (10) according to aspect 30,
wherein the display device (40) is adapted to display the recognition feature (50).

Aspect 32. The medical system (10) according to any of the preceding aspects, in particular according to any of aspects 23 to 31,
wherein a first device identification is assigned to the imaging instrument (12), wherein a second device identification is assigned to the display device (40), wherein an assignment rule is stored in the pairing unit (46) which defines combinations of different device identifications as permissible or impermissible, and wherein the pairing unit (46) is adapted to compare the first device identification with the second device identification using the assignment rule and to establish or prevent pairing of the imaging instrument (12) and the display device (40) in accordance with the application of the assignment rule.

Aspect 33. The medical system (10) according to any of the preceding aspects, in particular according to any of aspects 23 to 32,
wherein the security unit (48) is adapted to determine a user authorization of the user, to compare the user authorization with a predetermined required authorization, and to prevent the transmission of the image data from the imaging instrument (12) to the display device (40) if the user authorization does not reach the predetermined required authorization.

Aspect 34. The medical system (10) according to any of the preceding aspects, in particular according to any of aspects 23 to 33,
further comprising a storage unit (52) in which information about the pairing of the imaging instrument (12) with the display device (40) and/or the confirmation of the pairing by the user is stored.

Aspect 35. The medical imaging instrument (12) for a medical system (10) according to any of the preceding aspects, in particular according to any of aspects 23 to 34.

Aspect 36. The medical imaging instrument (12), in particular for a medical system (10) according to any of aspects 23 to 34, comprising:
a pairing unit (46) adapted to establish a pairing of the imaging instrument (12) with a display device (40), wherein, in a paired state, the image data is transmittable from the imaging instrument (12) to the display device (40); and
a security unit (48) adapted to recognize a confirmation of the pairing by the user, wherein the security unit (48) is adapted to prevent the transmission of the image data from the imaging instrument (12) to the display device (40) in the absence of the confirmation of the pairing by the user.

Aspect 37. The method of operating a medical system (10) according to any of aspects 23 to 34 and/or a medical imaging instrument (12) according to aspect 35 or 36.

Aspect 38. The method of connecting a medical imaging instrument (12) to a display device (40), in particular performed by means of a medical system (10) according to any of aspects 23 to 34 and/or a medical imaging instrument (12) according to aspect 35 or 36,
wherein the medical imaging instrument (12) is adapted to generate image data; and
wherein the display device (40) is adapted to receive image data and generate a representation (44) for a user;
comprising the steps of:
establishing a pairing of the imaging instrument (12) and the display device (40), wherein, in a paired state, the image data is transmittable from the imaging instrument (12) to the display device (40);
recognizing confirmation of pairing by the user; and preventing transmission of the image data from the imaging instrument (12) to the display device (40) in the absence of confirmation of pairing by the user.

Aspect 39. A medical system (10) comprising:
an object (54) which has a recognition feature (50) and which is identifiable by means of the recognition feature (50);
a medical imaging instrument (12) having a camera (56), by means of which images can be captured and image data can be generated, wherein an image of the recognition feature (50) of the object (54) can be captured by means of the camera (56);
a recognition unit (58) adapted to recognize the presence of the recognition feature (50) in the image of the recognition feature (50) captured by the camera (56) and to generate
a recognition signal in accordance with the recognition of the presence of the recognition feature (50); and
a control unit (60) adapted to process the recognition signal and to activate at least one function of the medical system (10) relating to the object (54) in accordance with the recognition signal.

Aspect 40. The medical system (10) according to aspect 39,
wherein the object (54) comprises a display device (40) adapted to receive image data and generate a representation (44) for a user.

Aspect 41. The medical system (10) according to aspect 40,
wherein the display device (40) is adapted to display the recognition feature (50).

Aspect 42. The medical system (10) according to any of the preceding aspects, in particular according to any of aspects 39 to 41,
wherein the action related to the object (54) is a pairing of the imaging instrument (12) with the display device (40).

Aspect 43. The medical system (10) according to any of the preceding aspects, in particular according to any of aspects 39 to 42,
wherein the medical imaging instrument (12) is adapted to capture the image (55) of the recognition feature (50) of the object (54) in a recognition mode and/or to generate associated image data such that at least image areas (62) which do not contain the recognition feature (50) are unrecognizable.

Aspect 44. The medical system (10) according to any of the preceding aspects, in particular according to any of aspects 39 to 43,
wherein the image areas (62) are unrecognizable due to defocusing of the camera (56).

Aspect 45. The medical system (10) according to any of the preceding aspects, in particular according to any of aspects 39 to 44,
wherein the recognition feature (50) is such that it is recognizable in an image of the recognition feature (50) captured in a defocused state of the camera (56).

Aspect 46. The medical system (10) according to any of the preceding aspects, in particular according to any of aspects 39 to 45,
wherein the recognition feature (50) comprises a barcode (64).

Aspect 47. The medical system (10) according to any of the preceding aspects, in particular according to any of aspects 39 to 46,
wherein the recognition feature (50) comprises a QR code (66).

Aspect 48. The medical system (10) according to any of the preceding aspects, in particular according to any of aspects 39 to 47,
wherein the recognition feature (50) comprises a color code (68).

Aspect 49. The medical system (10) according to any of the preceding aspects, in particular according to any of aspects 39 to 48,
wherein the recognition feature (50) comprises a temporal color sequence.

Aspect 50. The medical system (10) according to any of the preceding aspects, in particular according to any of aspects 39 to 49,
wherein the recognition feature (50) comprises a temporal light-dark sequence.

Aspect 51. The medical system (10) according to any of the preceding aspects, in particular according to any of aspects 39 to 50,
wherein the recognition feature (50) is linked to an instrument identification.

Aspect 52. The medical system (10) according to any of the preceding aspects, in particular according to any of aspects 39 to 51,
wherein the object (54) comprises a patient file (70).

Aspect 53. The medical system (10) according to any of the preceding aspects, in particular according to any of aspects 39 to 52,
wherein the object (54) comprises an item of clothing (72).

Aspect 54. The medical imaging instrument (12) for a medical system (10) according to any of the preceding aspects, in particular according to any of aspects 39 to 53.

Aspect 55. The medical imaging instrument (12), in particular for a medical system (10) according to any of aspects 39 to 53, in particular an endoscope (74), comprising:
a camera (56), by means of which images can be captured and image data can be generated;
a recognition unit (58) adapted to recognize the presence of a recognition feature (50) in an image of the recognition feature (50) captured by the camera (56) and to generate a recognition signal as a function of the recognition feature (50); and
a control unit (60) adapted to process the recognition signal and to activate at least one function of the medical imaging instrument (12) in accordance with the recognition signal.

Aspect 56. The method of operating a medical system (10) according to any of aspects 39 to 53 and/or a medical imaging instrument (12) according to aspect 54 or 55.

Aspect 57. The method using a medical system (10), in particular according to any of aspects 39 to 53,
wherein the medical system (10) comprises:
an object (54) which has a recognition feature (50) and which is identifiable by means of the recognition feature (50); and
a medical imaging instrument (12) having a camera (56), by means of which images can be captured and image data can be generated, wherein an image of the recognition feature (50) of the object (54) can be captured by means of the camera (56);
comprising the step:
capturing an image of the recognition feature (50) of the object (54) by means of the camera (56).

Aspect 58. The method according to aspect 57,
further comprising the steps:
recognizing the presence of the recognition feature (50) in the image of the recognition feature (50) captured by the camera (56);
generating a recognition signal in accordance with the recognition of the presence of the recognition feature (50); and
activating at least one function of the medical system (10) related to the object (54) in accordance with the recognition signal.

Aspect 59. The method according to aspect 57 or 58,
wherein the object (54) comprises a display device (40) adapted to receive image data and generate a representation for a user;
further comprising the step:
displaying the recognition feature (50) on the display device (40).

Aspect 60. A medical system (10) comprising:
a medical imaging instrument (12) adapted to generate image data and to wirelessly transmit the image data;
a display device (40) adapted to receive image data and generate a representation (44) for a user; and
a gesture recognition unit (76) adapted to recognize a gesture of the user directed towards the display device (40) and to establish a pairing between the imaging instrument (12) and the display device (40) in accordance with the recognition of the gesture.

Aspect 61. The medical system (10) according to aspect 60,
wherein the gesture recognition unit (76) is arranged on the display device (40).

Aspect 62. The medical system (10) according to aspect 60 or 61, further comprising:
a further display device (78) adapted to receive image data and generate a representation (44) for a user, wherein the further display device (78) is paired with the medical imaging instrument (12),
wherein the gesture recognition unit (76) is arranged to recognize the gesture of the user directed towards the display device (40) and, in accordance with the recognition of the gesture, to establish a pairing between the imaging instrument (12) and the display device (40) in addition to the pairing with the further display device (78) such that representations (44) based on the image data can be displayed both on the display device (40) and on the further display device (78).

Aspect 63. The medical system (10) according to aspect 62,
wherein the gesture recognition unit (76) is arranged on the further display device (78).

Aspect 64. The medical system (10) according to aspect 62 or 63,
wherein the imaging instrument (12) and the display device (40) can be paired such that the image data and/or information relating to the representation (44) can be transmitted from the further display device (78) to the display device (40).

Aspect 65. The medical system (10) according to any of the preceding aspects, in particular according to any of aspects 60 to 64,
wherein the gesture recognition unit (76) comprises at least one gesture recognition module (80) and/or can be expanded by connecting further gesture recognition modules (80).

Aspect 66. The medical system (10) according to any of the preceding aspects, in particular according to any of aspects 60 to 65, comprising:
a plurality of display devices (82) each adapted to receive image data and generate a representation (44) for a user,
wherein the gesture recognition unit (76) is arranged to recognize a gesture of the user directed towards a particular display device (40) of the display devices (82) and to establish a pairing between the imaging instrument (12) and the particular display device (40) of the display devices (82) in accordance with the recognition of the gesture directed towards the particular display device (40) of the display devices (82).

Aspect 67. The medical system (10) according to any of the preceding aspects, in particular according to any of aspects 60 to 66,
wherein the gesture recognition unit (76) comprises a stereoscopic camera system (84).

Aspect 68. The medical system (10) according to any of the preceding aspects, in particular according to any of aspects 60 to 67, further comprising:
a microphone (86) adapted to receive an audio signal,
wherein the gesture recognition unit (76) is adapted to recognize the audio signal and to prevent or allow pairing between the imaging instrument (12) and the display device (40) in accordance with the recognition of the audio signal.

Aspect 69. The method of operating a medical system (10) according to any of the preceding aspects, in particular according to any of aspects 60 to 68.

Aspect 70. The method of coupling a medical imaging instrument (12) to a display device (40), in particular to a medical system (10) according to any of aspects 60 to 68,
wherein the medical imaging instrument (12) is adapted to generate image data and to transmit the image data wirelessly; and
wherein the display device (40) is adapted to receive image data and generate a representation (44) for a user;
comprising the steps of:
recognizing a gesture of the user directed towards the display device (40); and
establishing a pairing between the imaging instrument (12) and the display device (40) in accordance with the recognition of the gesture.

1. A medical system comprising:
a medical imaging instrument adapted to generate image data and to wirelessly transmit the image data;
a display device adapted to receive image data and generate a representation for a user; and
a gesture recognition unit adapted to recognize a gesture of the user directed towards the display device and to establish a pairing between the imaging instrument and the display device in accordance with the recognition of the gesture.

2. The medical system according to claim 1, wherein the gesture recognition unit is arranged on the display device.

3. The medical system according to claim 1, further comprising:
a further display device adapted to receive image data and generate a representation for a user, wherein the further display device is paired with the medical imaging instrument,
wherein the gesture recognition unit is arranged to recognize the gesture of the user directed towards the display device and, in accordance with the recognition of the gesture, to establish a pairing between the imaging instrument and the display device in addition to the pairing with the further display device such that representations based on the image data can be displayed both on the display device and on the further display device.

4. The medical system according to claim 3, wherein the gesture recognition unit is arranged on the further display device.

5. The medical system according to claim 3, wherein the imaging instrument and the display device can be paired such that the image data and/or information relating to the representation can be transmitted from the further display device to the display device.

6. The medical system according to claim 1, wherein the gesture recognition unit comprises at least one gesture recognition module and/or can be expanded by connecting further gesture recognition modules.

7. The medical system according to claim 1, comprising:
a plurality of display devices each adapted to receive image data and generate a representation for a user,
wherein the gesture recognition unit is arranged to recognize a gesture of the user directed towards a particular display device of the display devices and to establish a pairing between the imaging instrument and the particular display device of the display devices in accordance with the recognition of the gesture directed towards the particular display device of the display devices.

8. The medical system according to claim 1, wherein the gesture recognition unit comprises a stereoscopic camera system.

9. The medical system according to claim 1, further comprising:
a microphone adapted to receive an audio signal,
wherein the gesture recognition unit is adapted to recognize the audio signal and to prevent or allow pairing between the imaging instrument and the display device in accordance with the recognition of the audio signal.

10. A method of operating a medical system according to claim 1.

11. A method of coupling a medical imaging instrument to a display device, in particular to a medical system according to claim 1, wherein the medical imaging instrument is adapted to generate image data and to transmit the image data wirelessly; and
wherein the display device is adapted to receive image data and generate a representation for a user;
comprising the steps of:
recognizing a gesture of the user directed towards the display device; and
establishing a pairing between the imaging instrument and the display device in accordance with the recognition of the gesture.
